SEQUENCE LISTING (9 sequences)

SEQ ID NO: 1 - DNA, 14543 bp, Homo sapiens
gaattcagttagttgactttttgtactttataagcgtgatgattgggtgttcccgtgtgagatgcgccaccctcgaaccttgttacgacgtcggcacattgcgcgtctgacatgaagaaaaaaaaaattcagttagtccaccaggcacagtggctaaggcctgtaatccctgcactttgagaggccaaggcaggaggatcacttgaacccaggagttcgagaccagcctaggcaacatagcgagactccgtttcaaacaacaaataaaaataattagtcgggcatggtggtgcgcgcctacagtaccaactactcgggaggctgaggcgagacgatcgcttgagccagggaggtcaaggctgcagtgagccaagctcgcgccactgcactccagcccgggcgacagagtgagaccctgtctccaaaaaaaaaaaaaaacaccaaaccttagaggggtgaaaaaaaattttatagtggaaatacagtaacgagttggcctagcctcgcctccgttacaacagcctacggtgctggaggatccttctgcgcacgcgcacagcctccggccggctatttccgcgagcgcgttccatcctctaccgagcgcgcgcgaagactacggaggtcgactcgggagcgcgcacgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtacgggcccggcgcttaggcccgacccagcagggacgatagcacggtctgaagcccctctaccgccccggggtccattttgcagacggggaactgaggccccttgaggcaggacacatccagggtgacgctgctcgtaagcgtcagagcattctttttttttttttttttttttttctgagacggagtctcgctctgtcgcccaggctggagtgcagtggcgcgatctcgactcactgcagcctccgcctcccgggttcaagcgattctcctgcctcagcctcctgagtagctgggattacaggcgtgcgccaccgcgcccggctgatttttatatttttagtagagacggggtttcaccatgttggtcaggctggtctcgaactaactgacctcgtgatccgcccgcctcggccttcccaaagtgctgggcttatgggcatgagcctccgcgcccggcccagagcattctttataaggccgaatagtttgcatttgaaggtggctcccccccagtcccccaccccacgtgtattttcccctcaaagaaaagctgcatccttaacaccccatctgttcagtcctcatgactccagtgggccagttctgcgtagtccctgccctcgtggagaacacattcctcctggggagactgacagatgcaaagacaggaacaagccagggtcatgttggcgccggaagagccgaccgtgtgtggcgtgggaaattgacttacctgcctgctgggagatggaggggttgcggttgtgtggtttcagttaaggagcacttcccggagaaggaagagagcaggatggagtaggaactagccaaccctaggtaagaggttctagacatgcgtgcgttgagacctggagtcttgggagaggatgcttaaaaggtgattttacccctaggaatatgggggcactgaaatttttttttttttttgagacgggagtcttgctctgcaagctggagtgcagtggcccacgctagaatgcagtggcgcgattgcggctcattgcaacatctgccacctggggtcaagtggttctcttgcctcagcctcccgaggagcggggattactggcgtgcgccaccactcctggctaattttttttttagtagagacgggggtttcgtcattttggctaggctggtctcgaactcctgacctcagatgatccacccgccttggcctcccaaagtgctgagattacaggtgtaagccactgcgcccagccctttgaaagtttttcagtatttatgtatatatatttttgagttggagtctggatctgtcgccagactggagtgctgttgcacaatcttggctcactgcaatctccgactccctggttcaagcgattctcctgcctcagcctcccaagtagctgggattacaggcacgcactaccattcccagctaattttttgtattcttagtagagacagggtttcaccatgttggccaggatggtctccatctcctgcgctcgtgatctgcctgcttcggcctcccaaagtgctgggattacaggcgtgctgggatttcggccacaacgtccgaccgaaagtttttaagcagggacatgacattgtcagatttatatactgaaaagctcacccaggttgccaagtggtttggaggggaaagactgctgtcgaggaagcagttaggtagttgtgaaaacccaggtgaggaataactaggccttacctaaggtgcaggcagtaatcttgccatggcctttaagcagagaagtagtcctagtgtcacttaatctttacaaaggatttttgcaaggatcccgatctttcttccttgagggtggtgtacttaatacacttttacaccagacttctaatgttagatgaagaacacagtatttccagggatcaacatttctgtaggctcctattttatataggaaattgtatgaattttgtattttactccaaaatttttctgtgcccgatttaatataaaaatttactgagcctgggtgcagtggctcatgcctgtgatctcagcactttgggaggctgaggcaggaggatttcttgagcccaggagctggagaccagcctgtgcaacatagtgagaccctgtctgtatttaaaaaaaaaaaaaaattcttgaaaaattagcagggcacattcctgcctttagtcccagctacttgggaagctgaggcaggaagatcacccgaacccaggagttggaggctacagtgaactatgatggtgcctctgaatagttgctgtactctagtctggtaacacagcaagaccctgtctctctatcttgtctttttttttttttttttttgagacaggatgtcctgctgttgcctgggctggagtgtggaggctggagtttggtggcatgatcacggctcattgcacccttaacctgggctcaagcagtcctcccagagcttcagcttcccaaagtagctgggactataggcatgctccactatgtctggctaatttctttttttatttttatttttagtagagatgaggtcttgctatgttgcccaggctgagacctcatctcttttttattttttttaaattttttattatactttaagttctagggtacatgtgcacaacgtgcaggtttgttacatatgtaaacatgtgccatgttggtgtgctgcacccatagagacctcgtcttaaaaaaagaaaataacattacttttgaaggtacttaatgcactgaattgtacatttaaaaatggttaaaatggtaaatgtttgaggcaggtagatccacctgaggtcaggagttcaagaccagcctgaccaatatggtggaaccctgtctctgctaaaaatacaaaagttagctgcatgtggtggcatgcgcctgtttagtcccagctactcgggaggctgaggcaggagaattgcttgaacctgggaggcggaggtggcagtgagccaagatcacaccactacactccagcctgggcaacagagcaagactccatctctaaataataaataaaatggtaacttttatgtatattttaccaaaatttaaaaaattacaagtttacatttcttaaaatttcccatcaaatctgtaagtaaatttatgccccgaggaacaagtgctatatttattctgagacaacctcctccttccttaaacagaatcttagggctggaggattgcttcctgccctcttttgtttgtgatgtatgcattttgaaaattctgggccgggcgcagtggctcactcctgtaatcccagcactttgggaggccgaggcgggcggatcacaagatcaggagattgagaccatcctggctaatacggtgaaaccctgtctctactgaaaataacaaaaaattagccgggcgtggtggcgggcacctgtggtcccagctatttgggaggctgtggcaggagaatggcataaacctgggaggcggagcttgcagtgagccgagatcgtgccactgcactccagcctgggcgacagagcgagactgcatctcaaaaaaaaaaaaaaagaaaaagaaaagaaaattctggtataatttacatacagtaaaatgcacagatcttagggtttgatgagttttctctcgacatgtttttgcacttccttgtttttgagaagcactgatttgagaagtcagtggctttttctctttagtttgcagggtttgctgtgatttgtaatcacgtacttgacctaggcttcccttttccaccatggtagcagaaagggcatgggatttagagctttaagtacgcgctctttgcttactgtcttataccttgagcatgtcacttctcctctcagacttgttttctcatctgtaaatggatctgttgtgaggactgactgagataatgttactagaagggctttgtataatatttaagcagagtgagaggtaagctttttgtgtaggtcaggggaaatggagaaaataggtgccctgactcagaccagtctggctctttttttttttttttttggagacggagtcttgctctgtcacccaggctggagtgcagtggcgcgatctcggctcacggcaagctccacctcctgggttcacaccattctcctgcctcagcctcccgagtagctgggactacaggcgctcgccacacacctggctaatttttttgtatttttagtagagacgaggtttcaccacgttagccaggacggtcttgatctcctgacctcatgatccgcctgcctcggcctcccaaagtgctgggattacaggtgtgggccaccgtgcccagccaccggtgtggctctttaacaacctttgcttgtcccgataggtcacctttggctcttcagagatgcagggacacacgatgggcttctggttaaccaaactgaattatttgtgccatctctcaatgttgacggacagcctatttttgccaatatcacactgccaggtactgacgttttactttttaaaaagataaggttgttgtggtaagtacaggatagaccacttgaaaaattaagcccagttctcaatttttgcctgatgtcaggcacggtatccaatctttttgtatcctattctctaccataaataaaatggaagtgatgtatttgtacgttatgtgttaaaggtgttatggtgtctcaaaagcactttgggctcttaagagacaagcgaaattaaagtatcatatcataggttagttttgtagaattgtagaattacgaatgccttttgtttccctggccaaattgtgccctggagttccaggagaacaatgtgtagagcatgagatattttggcttatttgttgctgacttctaattttttttatttttttgagacagaatctcgctgtgttagctaggctggagtgcagtggcgcaatctcggctcactgcaacctccgcctactgggttccagcgattctcttgtctcagcctcccgagtagctgggactacaggcgtgtgccacccactctgataattttttgtatttttagtagagacggggtttcaccgtgttagccaggatggtctccatctcctgacctcatgatctgcccgcctacgcctcccaaagtgctgggattacaggcatcagccacagcacctggcctatgtattttcaatttaacacaatcaagctcacagtgccaatcagaggtgttttttttttttttaatttttatttttagagagtctcacagtgtcatccaggctggagtgcagtggtgcgatttcagctcactgcaacctctgcatcctgggttcaagtgattctcctgcctcagcctcctgggtagctggggttataggtgcctgtcaccacacctggctaatttttgtatttttagtagagatgaggtttcaccatgttggccaggctgatcttgaactcctgacctcaggtgatctgcccacctcagcctcccaaagtgctgggattacaggcgtgagccactgcgtccagcctgtttttttttttttttaaatcattgaagattggtataatacttcactatttgtttgaagctcaaatgattttatcagggtaaaccctaataaactgatgttcctgtgggtaaaaaaaacctcactaaagaccagcagtgtgtggtggctcctgcctgtaatcatgcctgtaattccagcacttagggaggctatggcgggagggtcgcttgagaccaggagttcttgaccagcctggacaacaaagtgagaccccagctccacaaaaaaatttttttttaattacctgggcatcttagcatatgcctgtggtcacagctatttgggaggcttaggtgggaggatcccttgagcccaggagtttgaggctgcagtgagccatgatcataccactgcactccagcccaggtgacagagtgagatcctgtctcaaaaaaagaaaaaaaaaactcaaaaaccccccaaatacatgggtttcataggatccaaactactatgtgtgtatagatcctgttttaaggaagtagatatataaaaatgagcattgctaagttaaatttggtaaatttgccttatagaacaccctcgagtacgtttccagtgagtgtaaaataggaattgggatacccaattcagttgtactaaattttcttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggcctagattttctaagtacacattgttttggttatgtgttttgtgactaccaccccaaaactaataaccaccttttttttttttttgagacagagtctcactgtgtcacccaggctggagtgcagtggcgtgtgatcttggctcactgcaacctctgcctctcgggttcaagtgattctcctgcttcagcagctgggactacaggtgtgcaccaccaagcctggctaattttttgcattttagtggagacgggggtttcaccatgttgaccaggctgatcttaaactcttgagctcaggcagtctgcctgcctcagcctcctaaagtgctaggattacaggcgtgagccactgcgcccagcccaccgttttatttgttcataattctgtagtccaggctgggctcagctaggcagttactctgctggtggtagtcgttggtgtggctgccttttgctggcagctgggggctgggcctgtccctcttttttttttttctcttttcttttttcttttttttcaagatagggtctcactctgtcacccaggttggagtgcagtggcatgatcttagctcactgcaacctctgcctccagggctcaagtgatcctcccacctcagcctccccagtcgctgggaccacaggcatgtgccaccatgcctggctaattttttgtgtattttgtagagacggggtttcgccatgttgccaggctggtctcgaactcctgagctcaggcgatctactgacgttggcctcccaaagtgttgggatcacaggcatgaaccaccatgcctggccagggcctgttcctctttatgtggtctctctagcagggtagctcagggctttcaaaagtataaaagcagaagtcagcaggcctttttaaggcttcggcctagaattgccagtgtcgcttcatcgacattcagttagttaaagcaatcacaagcccagcccatttcaaggtgaaattactacagaggcatgaacaccatgaggtgtccatagggggccatcagcataacacactgccacatacatgcactcactttttttctttaacctaaagtgagatccatcagtagtacaggtagttgttggcaaagcctcttgttcgttccttgtactgagaccctagtctgtcactgaggatttggtttttgcccttccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttatataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgggatggagkctcactcttgtcacccaggctggagtgcagtggcgccatctcggctcactgcaacctctgcctcctgagttcacgtaatcctcctgagtagctgggattacaggtgcctgccaccacgctggccaatttttgtacttttagtagagacagtgtttcgccatgttggccaggctggtttcaaactcctgacctcaggtgatccgcccacctcagcctcccaaaatggtgggattacaggtgtgtgggccaccgtgcctggctgattcagcattttttatcaggcaggaccaggtggacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttgggaagccaaggtaagaggattgcttgagcccagaacttcaagatgagcctgggctcatagtgagaacccgtctatacaaaaaatttttaaaaattagcatggcggcacacatctgtaatcctagctacttggcaggctgaggtgagaagatcattggagtttaggaattggaggcggcagtgagtcatgagtatgccgctgcactccagcctgggggacagagcaagaccctgcctcaaaaaaaaaaaaaaaaaaaattcaggccgggaatggtggttcacgcctgtaatcccagcactttggggggtcgaggtgggcagatcacctgaggtcaggagttcgagaccagcctggccaacatggtaaaaccccatttctactaaaaaatacaagaattagctgggtgtggtggcgcatgcctgtaatcctagctactcaggaggctgaggcaggagaatcacttgaccccaggaggcgaagattgcagtgagctgatatcgcaccattgtactccagcctgtgtgacagagcaatactcttgtcccaaaaaaaaaaaaaattcaaatcagagtgaagtgaatgagacactccagttttccttctactccgaattttagctcctcctttcaacattcaacaaatagtcttttttttttttttttttttttggggatggagtctccctctgttgcccaggctggagtgcagaggtgcgatctctgctcactacaagctctgcctcccgagttcaagtgattctcctggctcaccctcctgagctgggattacaggcgcctgccaccatgcctggctaattttgtgtttttagtggagacggggtttcaccatgttgtccaggatggtcttgatctcctgaccttgtgatccacccacctcagcctcccaaagtggtgggattacaggtgtgagccaccgcgtccagccagctttattattttttttaagctgtctttgtgtcaaaatgatagttcatgctcctcttgttaaaacctgcaggccgagcacagtggctcatgcctgtaatcccagcattttgggagaccaaggcggatggatcacctgaggtcaggagctcaagaccagcctggctaacatggtgaaacctcatctccacttaaaatacaaaaattgccggccgcggcggctcatgcctgtaatcccagcactttgggaggcctaggcgggtggatcacgacgtcaggaaatcgagaccatcctggctaacacgggtgaaaccccgtctctattaaaaaatagaaaaaattaggcgggcgtggtggtgagcgcctgtagtcccagctactcgagagcctgaggcaggagaatggcatgaacctggaaggtggagcttgcagtgagctgagatggtgccactgcactctaacctgggcgacagagtgagactccgtctcaaaaaaaaaaacaaaaaccaaaacttatccaggtgtggcggtgggcgcctgtgaggcaggcgaatctcttgaacccgggaggcggaggttgcagtgagccaagatcacaccattgcactccagcctgggaaacaagagtgaaattccatctcaaaaccaaattttcaaaaaaaaaacatgccgcttgagtactgtgtttttggtgttgtccaaggaaaattaaaacctgtagcatgaataatgtttgttttcatttcgaatcttgtgaatgtattaaatatatcgctcttaagagacggtgaagttcctatttcaagttttttttgttttgttttgtttttaagctgttttttaatacattaaatggtgctgagtaaaggaaataggcagggtgtgttgtgtggtgttttaactaggcgcttctctctcagagagttttgaaacctgtttacataaaggcccaagatgggaaggagatccaaacataagccaccagcctcattccaagtctcttctctttccaaccctggattttttttttttatttaacattgtttcttttagctttatttttcttataaaagaaatgtatcactataaaaaattacacactacagaaaaatattaagaagaaaaacattcacatcggaaacaaagttttttcccatgaaaacagaacccaaaagggtaagtggttagtatttcaccagcaattatgttgagaataaggccaggcgaggtggctcacgcctgtaatctcagcactttgggaggccagggcaggcagatcatctgaggtcaggagtttgagaccagcctggccaacatggtgaaaccctatctctactaaaaattaaaaaattagctgggtgtggtggcatgtacctgtaatcccagctattcaggaggctgaggcaggagaattgcttgaacctgggaggcggaggttgcagtgagctgagattgcaccattgcactctagcctgggcaacgagtgaaactccgtctcaaaagaaaaaaatatatatatatagagagagagagagagagaataccacagtgagggcatgggctagaaatcagtgcactaaggatatgaaatagatgtcaatgtgaacttttcggatactttgaccctgggtctttgtatcctcttcttagcacctcagtcccacgctctgctagtcattggcttcctgataccccttcaatacagactgagtatccctaatccaaaaatttcaaatccaaagcactccaaaatccaagagtccaacgtgacgccacaagtggaaagttccacatgcgagtacttaacacaaacttgtttcacgtgcaaaactggggaaaatattgcttacaattacctacagcctgtgtctataaggtgtttatgaaactggtgttatgatgtatatgttttctttttttgttcctggctcataactcccatagcccttgttacggatgtgagccaccttgcctggctgatttttaagttttttgtagagatggggtctcgctgtgttgccctggctggttttaactcctgggctcaagcgatcctcccaccttggcctcccaaagccctgggattacaggtgagattacaaccctcatttcagagaaggtcctaccccataccctgggggaaggaatggtgacatcataaagcctcgttaaaacccatgaggacagtggagagtgtcaggatagctgaactacgtgtagaggttcctggagggtggtgcgcccagggaggggacagaagctctgcgccccttatcccataccttggtgtacgcatctcttcatctgtatccttcgtaatatcctttatgataaaccaggtaggccgggcgtggtggctcacacatataatcccagcactttgggaggctgaggtaggaggattgcttcagcctgggagttcaagataacatcatagtgagatcctgtctctactagaaaaaaaaagaacaaccaggagtggtggcgcatgcttgcagtcccagctgttcagtttgcactccagcctgggagacagagcaagacctgctgtctcaaaaaaaaaagactggtaaacatttttcactgagttctgttagccactccagcaaattaaacccaaagcgaaggtggtgggaaccccaacttgaagctggttggtcagaagttctggagccctaaacttgctactggtgtgtgggtgggggcagtcttggggactgaggcctcaacctgcaggatctgatattatttccagaaagatggtgttggaagtgaattagaggatacctaattggtgttcactgcagaattgattgcttgctcgctctcgggaagaaatctacacatttggacacgaaagtgttctgggttggtattgtgttagtgtggaatctaga

SEQ ID NO: 2 - DNA, 20 bp, Artificial Sequence
  Primer
ccaaaccttagaggggtgaa

SEQ ID NO: 3 - DNA, 18 bp, Artificial Sequence
  Primer
ctccgcgatccagaccac

SEQ ID NO: 4 - DNA, 10 bp, Artificial Sequence
  Binding Sequence
rttacryaat

SEQ ID NO: 5 - DNA, 10 bp, Artificial Sequence
  Binding Sequence
gttacrtnan

SEQ ID NO: 6 - DNA, 18 bp, Artificial Sequence
  Binding Sequence
trtnnmttrcmnmanwcn

SEQ ID NO: 7 - DNA, 19 bp, Artificial Sequence
  Binding Sequence
raccacagatatccntgtt

SEQ ID NO: 8 - DNA, 13 bp, Artificial Sequence
  Binding Sequence
gngtgggsgcgns

SEQ ID NO: 9 - DNA, 13 bp, Artificial Sequence
  Binding Sequence
sncgcscccacnc

(19) United States
(12) Patent Application Publication
(10) Pub. No.: US 2004/0166491 A1
(43) Pub. Date: Aug. 26, 2004

(54) Vhl promoter diagnostic polymorphism

(76) Inventor: Lee A Henderson, Ithaca, NY (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/344,908

(22) PCT Filed: Aug. 9, 2001
(86) PCT No.: PCT/US01/24985

(51) Int. Cl. C12Q 1/68

(52) U.S. Cl. 435/6

(57) ABSTRACT

Disclosed is a single nucleotide polymorphism (SNP) associated with colon cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, alcoholic cirrhosis, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, end stage renal disease due to non-insulin dependent diabetes mellitus, and seizure disorder. Also disclosed are methods for using the SNP to determine susceptibility to these diseases; nucleotide sequences containing the SNP; kits for determining the presence of the SNP; and methods of treatment or prophylaxis based on the presence of the SNP.

### BACKGROUND

This invention relates to detection of individuals at risk for pathological conditions based on the presence of single nucleotide polymorphisms (SNPs) at positions 520 and 638 on the human von Hippel-Lindau syndrome tumor suppressor gene (vHL) promoter.

During the course of evolution, spontaneous mutations appear in the genomes of organisms. It has been estimated that variations in genomic DNA sequences are created continuously at a rate of about 100 new single base changes per individual (Kondrashow, J. Theor. Biol, 175:583-594, 1995; Crow, Exp. Clin. Immunogenet., 12:121-128, 1995). These changes, in the progenitor nucleotide sequences, may confer an evolutionary advantage, in which case the frequency of the mutation will likely increase, an evolutionary disadvantage in which case the frequency of the mutation is likely to decrease, or the mutation will be neutral. In certain cases, the mutation may be lethal in which case the mutation is not passed on to the next generation and so is quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphisms are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome is single nucleotide polymorphisms or SNPs. SNPs account for approximately 90% of human DNA polymorphism (Collins et al., Genome Res., 8:1229-1231, 1998). SNPs are single base pair positions in genomic DNA at which different sequence alternatives. (alleles) exist in a population. In addition, the least frequent allele must occur at a frequency of 1% or greater. Several definitions of SNPs exist in the literature (Brooks, Gene, 234:177-186, 1999). As used herein, the term “single nucleotide polymorphism” or “SNP” includes all single base variants and so includes nucleotide insertions and deletions in addition to single nucleotide substitutions (e.g. A->G). Nucleotide substitutions are of two types. A transition is the replacement of one purine by another purine or one pyrimidine by another pyrimidine. A transversion is the replacement of a purine for a pyrimidine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998). The frequency of SNPs varies with the type and location of the change. In base substitutions, two-thirds of the substitutions involve the C<->T (G<->A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct, as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect, where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769776, 1998. SNPs can be detected by restriction fragment length polymorphism (RFLP) (U.S. Pat. Nos. 5,324,631; 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases has also been used to detect SNPs.

There is growing recognition that SNPs can provide a powerful tool for the detection of individuals whose genetic make-up alters their susceptibility to certain diseases. There are four primary reasons why SNPs are especially suited for the identification of genotypes which predispose an individual to develop a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers but as candidates for gene therapy treatments to cure or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

### SUMMARY

The present inventor has discovered novel single nucleotide polymorphisms (SNPs) associated with the development of various diseases, including colon cancer, hypertension (HTN), atherosclerotic peripheral vascular disease due to hypertension (ASPVD due to HTN), cerebrovascular accident due to hypertension (CVA due to HTN), cataracts due to hypertension (cataracts due to HTN), cardiomyopathy with hypertension (HTN CM), myocardial infarction due to hypertension (MI due to HTN), end stage renal disease due to hypertension (ESRD due to HTN), non-insulin dependent diabetes mellitus (NIDDM), atherosclerotic peripheral vascular disease due to non-insulin dependent diabetes mellitus (ASPVD due to NIDDM), cerebrovascular accident due to non-insulin dependent diabetes mellitus (CVA due to NIDDM), ischemic cardiomyopathy (ischemic CM), ischemic cardiomyopathy with non-insulin dependent diabetes mellitus (ischemic CM with NIDDM), myocardial infarction due to non-insulin dependent diabetes mellitus (MI due to NDDM), atrial fibrillation without valvular disease (afib without valvular disease), alcohol abuse, alcoholic cirrhosis, anxiety, asthma, chronic obstructive pulmonary disease (COPD), cholecystectomy, degenerative joint disease (DJD), end stage renal disease and frequent de-clots (ESRD and frequent de-clots), end stage renal disease due to focal segmental glomerular sclerosis (ESRD due to FSGS), end stage renal disease due to non-insulin dependent diabetes mellitus (ESRD due to NIDDM), end stage renal disease due to insulin dependent diabetes mellitus (ESRD due to IDDM), or seizure disorder. As such, these polymorphisms provide a method for diagnosing a genetic predisposition for the development of these diseases in individuals. Information obtained from the detection of SNPs associated with the development of these diseases is of great value in their treatment and prevention.

Accordingly, one aspect of the present invention provides a method for diagnosing a genetic predisposition for colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder in a subject, comprising obtaining a sample containing at least one polynucleotide from the subject, and analyzing the polynucleotide to detect the genetic polymorphism wherein the presence or absence of said genetic polymorphism is associated with an altered susceptibility to developing colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder. In one embodiment, the polymorphism is located in the vHL gene.

Another aspect of the present invention provides an isolated nucleic acid sequence comprising at least 10 contiguous nucleotides from SEQ ID NO: 1, or their complements, wherein the sequence contains at least one polymorphic site associated with a disease and in particular colon cancer, HTN, ASPNTD due to HTN, CVA due to HTN, cataracts due to HTN, HTN CM, MI due to HTN, ESRD due to HTN, NIDDM, ASPVD due to NIDDM. CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder. Yet another aspect of the invention is a kit for the detection of a polymorphism comprising, at a minimum, at least one polynucleotide of at least 10 contiguous nucleotides of SEQ ID NO: 1, or their complements, wherein the polynucleotide contains at least one polymorphic site associated with colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder.

Yet another aspect of the invention provides a method for treating colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NDDM, ESRD due to IDDM, or seizure disorder comprising, obtaining a sample of biological material containing at least one polynucleotide from the subject; analyzing the polynucleotides to detect the presence of at least one polymorphism associated with colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder; and treating the subject in such a way as to counteract the effect of any such polymorphism detected.

Still another aspect of the invention provides a method for the prophylactic treatment of a subject with a genetic predisposition to colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder comprising, obtaining a sample of biological material containing at least one polynucleotide from the subject; analyzing the polynucleotide to detect the presence of at least one polymorphism associated with colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, Ischemic CM, Ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder; and treating the subject.

Further scope of the applicability of the present invention will become apparent from the detailed description and drawings provided below. It should be understood, however, that the following detailed description and examples, while indicating preferred embodiments of the invention, are given by way of illustration only, since various changes and modifications within the spirit and scope of the invention will become apparent to those skilled in the art from the following detailed description

### BRIEF DESCRIPTION OF THE DRAWINGS

These and other features, aspects, and advantages of the present invention will become better understood with regard to the following description, appended claims, and accompanying drawings where:

FIG. 1 shows SEQ ID NO: 1, the nucleotide sequence of the vHL gene as contained in GenBank Accession Number AF010238. Thus, all nucleotides will be positively numbered, rather than bear negative numbers reflecting their position upstream from the RNA polymerase II binding site (a TATA box in about half of eukaryotic genes), the transcription initiation site (a variable number of nucleotides downstream of, i.e. 3′ to, the TATA box), the translation start site, or the first codon of the encoded protein (the “A” of the “ATG” codon for methionine, the first amino acid of every eukaryotic protein). Since not all genes are fully annotated, and not all promoter sequences contain elements far downstream such as the “ATG” encoding the first methionine in the translated protein, we feel that the numbering system used in this patent application is the least troublesome.

The various numbering systems can be easily interconverted, if desired. According to the annotation of Accession Number AF 010238, the transcription start site and first exon both begin at position 643. The position of the “A” of the ATG codon for the first amino acid (methionine) of the protein, i.e. the translation start site, is at position 715.

The first SNP, C638->T is located at position 638 of the GenBank Accession Number AF 010238. The 20 nucleotides surrounding the SNP are as follows: 5′-G ACT CGG GAG [C/T] GCG CAC GCA G-3′ (nucleotides 628-648 of SEQ ID NO. 1). The C638->T SNP thus corresponds to position 638-643=−5 with reference to the transcription start site, and to position 638-715=−77 with reference to the first encoded “ATG” codon.

The second SNP, C638->T is located at position 638 of the GenBank Accession Number AF 010238. The 20 nucleotides surrounding the SNP are as follows: 5′-G ACT CGG GAG [C/T] GCG CAC GCA G-3′ (nucleotides 628-648 of SEQ ID NO. 1). The C638->T SNP thus corresponds to position 638-643=−5 with reference to the transcription start site, and to position 638-715=−77 with reference to the first encoded “ATG” codon.

### DEFINITIONS

nt=nucleotide

bp=base pair

kb=kilobase; 1000 base pairs

AFIB=atrial fibrillation without valvular disease

ASPVD=atherosclerotic peripheral-vascular disease

COPD=chronic obstructive pulmonary disease

CVA=cerebrovascular accident

DJD=degenerative joint disease, also know as osteoarthritis

DOL=dye-labeled oligonucleotide ligation assay

ESRD=end-stage renal disease

HTN=hypertension

MASDA=multiplexed allele-specific diagnostic assay

MADGE=microtiter array diagonal gel electrophoresis

MI=myocardial infarction

NIDDM=noninsulin-dependent diabetes mellitus

OLA=oligonucleotide ligation assay

PCR=polynierase chain reaction

RFLP=restriction fragment length polymorphism

SNP=single nucleotide polymorphism

“Polynucleotide” and “oligonucleotide” are used interchangeably and mean a linear polymer of at least 2 nucleotides joined together by phosphodiester bonds and may consist of either ribonucleotides or deoxyribonucleotides.

“Sequence” means the linear order in which monomers occur in a polymer, for example, the order of amino acids in a polypeptide or the order of nucleotides in a polynucleotide.

“Polymorphism” refers to a set of genetic variants at a particular genetic locus among individuals in a population.

“Promoter” means a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) coding region, as well as intervening non-coding sequences (“introns”) between individual coding segments (“exons”). A promoter is herein considered as a part of the corresponding gene. Coding refers to the representation of amino acids, start and stop signals in a three base ‘triplet’ code. Promoters are often upstream (“5“to”) the transcription initiation site of the gene.

“Gene therapy” means the introduction of a functional gene or genes from some source by any suitable method into a living cell to correct for a genetic defect.

“Reference allele” or “reference type” means the allele designated in the Gen Bank sequence listing for a given gene, in this case Gen Bank Accession Number AS 010238 for the vHL gene.

“Genetic variant” or “variant” means a specific genetic variant which is present at a particular genetic locus in at least one individual in a population and that differs from the reference type.

As used herein the terms “patient” and “subject” are not limited to human beings, but are intended to include all vertebrate animals in addition to human beings.

As used herein the terms “genetic predisposition”, “genetic susceptibility” and “susceptibility” all refer to the likelihood that an individual subject will develop a particular disease, condition or disorder. For example, a subject with an increased susceptibility or predisposition will be more likely than average to develop a disease, while a subject with a decreased predisposition will be less likely than average to develop the disease. A genetic variant is associated with an altered susceptibility or predisposition if the allele frequency of the genetic variant in a population or subpopulation with a disease, condition or disorder varies from its allele frequency in the population without the disease, condition or disorder (control population) or a control sequence (reference type) by at least 1%, preferably by at least 2%, more preferably by at least 4% and more preferably still by at least 8%. Alternatively, an odds ratio of 1.5 was chosen as the threshold of significance based on the recommendation of Austin et al. in Epidemiol. Rev., 16:65-76, 1994. “[E]pidemiology in general and case-control studies in particular are not well suited for detecting weak associations (odds ratios<1.5).” Id. at 66.

As used herein “isolated nucleic acid” means a species of the invention that is the predominate species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

As used herein, “allele frequency” means the frequency that a given allele appears in a population.

### DETAILED DESCRIPTION

All publications, patents, patent applications and other references cited in this application are herein incorporated by reference in their entirety as if each individual publication, patent, patent application or other reference were specifically and individually indicated to be incorporated by reference.

Novel Polymorphisms

The present application provides single nucleotide polymorphisms (SNPs) in a gene associated with colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, and seizure disorder. These polymorphisms consist of an A to G transition found in the vHL promoter at position 520 and a C to T transition at position 638 of the same promoter. These SNPs are further disclosed in Table 13.

Preparation of Samples

The presence of genetic variants in the above genes or their control regions, or in any other genes that may affect susceptibility to disease is determined by screening nucleic acid sequences from a population of individuals for such variants. The population is preferably comprised of some individuals with the disease of interest, so that any genetic variants that are found can be correlated with disease. The population is also preferably comprised of some individuals that have known risk for the disease. The population should preferably be large enough to have a reasonable chance of finding individuals with the sought-after genetic variant. As the size of the population increases, the ability to find significant correlations between a particular genetic variant and susceptibility to disease also increases.

The nucleic acid sequence can be DNA or RNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid must be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably is by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos. 4,965,188, 4,800,159, 4,683,202, and 4.683.195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR) (see, Wu and Wallace, Genomics, 4:560-569, 1989; Landegren et al., Science, 241:1077-1080, 1988), transcription amplification (Kwoh et al., Proc. Natl. Acad. Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factors for the disease. The populations should preferably be large enough to have a reasonable chance to find correlations between a particular genetic variant and susceptibility to the disease of interest. In addition, the allele frequency of the genetic variant in a population or subpopulation with the disease or pathology should vary from its allele frequency in the population without the disease or pathology (control population) or the control sequence (reference type) by at least 1%, preferably by at least 2%, more preferably by at least 4% and more preferably still by at least 8%.

Determination of unknown genetic variants, and in particular SNPs, within a particular nucleotide sequence among a population may be determined by any method known in the art, for example and without limitation, direct sequencing, restriction length fragment polymorphism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, 3^{rd} ed., Wiley, 1995 and Sambrook et al., Molecular Cloning, 2nd ed., Chap. 13, Cold Spring Harbor Laboratory Press, 1989. Sequencing can be cared out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

RFLP analysis (see, e.g. U.S. Pat. Nos. 5,324,631 and 5.645.995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in <220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.). Double strands are first heat-denatured. The single strands are then subjected to polyacrylamide gel electrophoresis under non-denaturing conditions at constant temperature (i.e., low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled reference type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET) (Keen et al., Trends Genet. 7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM) is based on the chemical reactivity of thymine (T) when mismatched with cytosine, guanine or thymine and the chemical reactivity of cytosine (C) when mismatched with thymine, adenine or cytosine (Cotton et al., Proc. Natl. Acad. Sci. USA, 85:4397-4401, 1988). Duplex DNA formed by hybridization of a reference type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the reference type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998, and will only be briefly reviewed here.

One type of assay has been termed an array hybridization assay, an example of which is the multiplexed allele-specific diagnostic assay (MASDA) (U.S. Pat. No. 5,834,181; Shuber et al., Hum. Molec. Genet., 6:337-347, 1997). In MASDA, samples from multiplex PCR are immobilized on a solid support. A single hybridization is conducted with a pool of labeled allele specific oligonucleotides (ASO). Any ASOs that hybridize to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end, such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during the PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can be achieved by Microtiter Array Diagonal Gel Electrophoresis (MADGE) (Day and Humphries, Anal. Biochem., 222:389-395, 1994). In this assay restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) (Sommer et al., Mayo Clin. Proc., 64:1361-1372 1989; Sarker et al., Anal. Biochem., 1990), allele-specific amplification (ASA), allele-specific PCR, and amplification refractory mutation system (ARMS) (Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determined whether an individual is homozygous or heterozygous for the mutation (Dutton and Sommer, BioTechniques, 11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers has a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can be used as a template.

In one method for mass screening for SNPs based on the OLA, amplified DNA templates are analyzed for their ability to serve as templates for ligation reactions between labeled oligonucleotide probes (Samotiaki et al., Genomics, 20:238-242, 1994). In this assay, two allele-specific probes labeled with either of two lanthamide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J. Hum. Genet., 52:46-59, 1993. In this method the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled dNTP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al., Human Mut., 7:34&354, 1996). In this method, incorporation of the ddNTP is determined using an automatic gel sequencer.

Minisequencing has also been adapted for use with microarrays (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21, January, 1999. PCR products are spotted on the array and allowed to anneal. The extension (elongation) reaction is carried out using a polymerase, a labeled DNTP and noncompeting ddNTPs. Incorporation of the labeled dNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with rhinisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease. In this method, a biological sample is obtained from a subject. The subject can be a human being or any vertebrate animal. The biological sample must contain polynucleotides and preferably genomic DNA. Samples that do not contain genomic DNA, for example, pure samples of mammalian red blood cells, are not suitable for use in the method. The form of the polynucleotide is not critically important such that the use of DNA, cDNA, RNA or mRNA is contemplated within the scope of the method. The polynucleotide is then analyzed to detect the presence of a genetic variant where such variant is associated with an increased risk of developing a disease, condition or disorder, and in particular colon cancer, HTN, ASPVD due to HTN, CVA due to HTN, cataracts due to HTN, HTN CM, CM due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder. In one embodiment, the genetic variant is located at one of the polymorphic sites contained in Table 13. In another embodiment, the genetic variant is one of the variants contained in Table 13 or the complement of any of the variants contained in Table 13. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1, or the complements of SEQ ID NO: 1. In one preferred embodiment, the sequence contains at least one polymorphic site associated with a disease, and in particular colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, ischemic CM, ischemic CM with NIDDM, Mf due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder. In one embodiment, the genetic variant is located at one of the polymorphic sites contained in Table 13. In another embodiment, the genetic variant is one of the variants contained in Table 13 or the complement of any of the variants contained in Table 13. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, and in particular colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder. The kits contain, at a minimum, at least one polynucleotide of at least 10 contiguous nucleotides of SEQ ID NO 1, or the complements of SEQ ID NO: 1. In one embodiment, the polynucleotide contains at least one polymorphic site, preferably, the polymorphic site is located at one of the sites contained in Table 13. Alternatively the 3′ end of the polynucleotide is immediately 5′ to a polymorphic site, preferably a polymorphic site located at one of the sites contained in Table 13. In one embodiment, the polymorphic site contains a genetic variant as denominated in Table 13, or the complement of any of the variants contained in Table 13. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder and in particular colon cancer, HTN, ASPVD due to HTN, CVA due to HTN, cataracts due to HTN, HTN CM, MI due to HTN, ESRD due to KIN, NIDDM, ASPVD due to NIDDM, CVA due to NIDDM, ischemic CM, ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder, caused either directly or indirectly by the presence of one or more single nucleotide polymorphisms. In this method genetic material from a patient, for example, DNA, cDNA, RNA or mRNA is screened for the presence of one or more SNPs associated with the disease of interest. Depending on the type and location of the SNP, a treatment regime is designed to counteract the effect of the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art. Once designed, the gene can be placed in the individual by any suitable means known in the art (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have an increased susceptibility to a disease, condition or disorder, and in particular colon cancer, HTN, ASPVD due to HTN, CVA due to HTN, cataracts due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder due to the presence of one or more single nucleotide polymorphisms. In this embodiment genetic material, such as DNA, cDNA, RNA or mRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

### EXAMPLES

Position of the single nucleotide polymorphism (SNP) is given according to the numbering scheme in GenBank Accession Number AF010238. Thus, all nucleotides will be positively numbered, rather than bear negative numbers reflecting their position upstream from the transcription initiation site, a scheme often used for promoters. The two numbering systems can be easily interconverted, if necessary. GenBank sequences can be found at http://wvw.ncbi.nlm.nih.gov/

In the following examples, SNPs are written as “reference sequence nucleotide”→“variant nucleotide.” Changes in nucleotide sequences are indicated in bold print. The standard nucleotide abbreviations are used in which A=adenine, C=cytosine, G=guanine, T-thymine, M=A or C, R=A or G, W=A or T, S=C or G, Y=C or T, K=G or T, V=A or C or G, H=A or C or T; D=A or G or T; B=C or G or T; N=A or C or G or T.

### Example 1

### Detection of Novel Polymorphisms by Direct Sequencing of Leukocyte Genomic DNA

Leukocytes were obtained from human whole blood collected with EDTA as an anticoagulant. Blood was obtained from a group of black men, black women, white men, and white women without any known disease. Blood was also obtained from individuals with colon cancer, HTN, ASPVD due to HTN, CVA due to HTN, cataracts due to HTN, HTN CM, MI due to HTN, BSRD due to NIDDM, NIDDM, ASPVD due to NIDDM, CVA due to NIDDM, ischemic CM, ischemic CM with NIDDM, MI due to NIDDM, afib without valvular disease, alcohol abuse, alcoholic cirrhosis, anxiety, asthma, COPD, cholecystectomy, DJD, ESRD and frequent de-clots, ESRD due to FSGS, ESRD due to NIDDM, ESRD due to IDDM, or seizure disorder as indicated in the tables below.

Genomic DNA was purified from the collected leukocytes using standard protocols well known to those of ordinary skill in the art of molecular biology (Ausubel et al., Short Protocol in Molecular Biology, 3^{rd} ed., John Wiley and Sons, 1995; Sambrook et al., Molecular Cloning, Cold Spring Harbor Laboratory Press, 1989; and Davis et al., Basic Methods in Molecular Biology, Elsevier Science Publishing, 1986). One hundred nanograms of purified genomic DNA were used in each PCR reaction.

Standard PCR reaction conditions were used. Methods for conducting PCR are well known in the art and can be found, for example, in U.S. Pat. Nos. 4,965,188, 4,800,159, 4,683,202, and 4.683.195; Ausbel et al., eds., Short Protocols in Molecular Biology, 3^{rd} ed., Wiley, 1995; and Innis et al., eds., PCR Protocols, Academic Press, 1990. The sense primer was 5′-CCA AAC CTT AGA GGG GTG AA-3′ (SEQ ID NO: 2). The anti-sense primer was 5′-CTC CGC GAT CCA GAC CAC-3′ (SEQ ID NO: 3). The PCR product produced spanned positions 441 to 711 of the human vHL gene (SEQ ID NO: 1).

The PCR reaction contained a total volume of 20 microliters (μl), consisting of 10 μl of a premade PCR reaction mix (Sigma “JumpStart Ready Mix with RED Taq Polymerase”). Primers at 10 μM were diluted to a final concentration of 0.3 μM in the PCR reaction mix. Approximately 25 ng of template leukocyte genomic DNA was used for each PCR amplification. Twenty-five microliters of an aqueous solution of genomic DNA (1 ng/ul) was dispensed to the wells of a 96-well plate, and dried down at 70° C. for 15 min. The DNA was rehydrated with 7 μl of ultra-pure but not autoclaved water (Milli-Q, Millipore Corp.). PCR conditions were as follows: 5 min at 94° C., followed by 45 cycles, where each cycle consisted of 94° C. for 45 seconds to denature the double-stranded DNA, then 64° C. for 45 seconds for specific annealing of primers to the single-stranded DNA, then 72° C. for 45 seconds for extension. After the 45th cycle, the reaction mixture was held at 72° C. for 10 min for a final extension reaction.

Post-PCR clean-up was performed as follows. PCR reactions were cleaned to remove unwanted primer and other impurities such as salts, enzymes, and unincorporated nucleotides that could inhibit sequencing. One of the following clean-up kits was used: Qiaquick-96 PCR Purification Kit (Qiagen) or Multiscreen-PCR Plates (Millipore, discussed below).

When using the Qiaquick protocol, PCR samples were added to the 96-well Qiaquick silica-gel membrane plate and a chaotropic salt, supplied as “TB Buffer,” was then added to each well. The PB Buffer caused the DNA to bind to the membrane. The plate was put onto the Qiagen vacuum manifold and vacuum was applied to the plate in order to pull sample and PB Buffer through the membrane. The filtrate was discarded. Next, the samples were washed twice using “PE Buffer.” Vacuum pressure was applied between each step to remove the buffer. Filtrate was similarly discarded after each wash. After the last PE Buffer wash, maximum vacuum pressure was applied to the membrane plate to generate maximum airflow through the membrane in order to evaporate residual ethanol left from the PE Buffer. The clean PCR product was then eluted from the filter using “EB Buffer.” The filtrate contained the cleaned PCR product and was collected. All buffers were supplied as part of the Qiaquick-96 PCR Purification Kit. The vacuum manifold was also purchased from Qiagen for exclusive use with the Qiaquick-96 Purification Kit.

When using the Millipore Multiscreen-PCR Plates, PCR samples were loaded into the wells of the Multiscreen-PCR Plate and the plate was then placed on a Millipore vacuum manifold. Vacuum pressure was applied for 10 minutes, and the filtrate was discarded. The plate was then removed from the vacuum manifold and 1100 μl of Milli-Q water was added to each well to rehydrate the DNA samples. After shaking on a plate shaker for 5 minutes, the plate was replaced on the manifold and vacuum pressure was applied for 5 minutes. The filtrate was again discarded. The plate was removed and 60 μl Milli-Q water was added to each well to again rehydrate the DNA samples. After shaking on a plate shaker for 10 minutes, the 601 μl of cleaned PCR product was transferred from the Multiscreen-PCR plate to another 96-well plate by pipetting. The Millipore vacuum manifold was purchased from Millipore for exclusive use with the Multiscreen-PCR plates.

The SNP typing for the disease associations provided in the first table in each category below (HTN, ESRD due to HTN, NIDDM, ESRD due to NIDDM, also known as the “Group I diseases”) was accomplished through a method called cycle sequencing. Cycle sequencing was performed on the clean PCR product using an ABI Prism Big Dye Terminator Cycle Sequencing Ready Reaction kit (Perkin-Elmer). For a total volume of 20 μl, the following reagents were added to each well of a 96-well plate: 2.0 μl Terminator Ready Reaction mix, 3.0 μl 5×Sequencing Buffer (ABI), 5-10 μl template (30-90 ng double stranded DNA), 3.2 pM primer primer used was the forward primer from the PCR reaction), and Milli-Q water to 20 μl total volume. The reaction plate was placed into a Hybaid thermal cycler block and programmed as follows: X 1 cycle: 1 degree/sec thermal ramp to 94° C., 94° C. for 1 min; X 35 cycles: 1 degree/sec thermal ramp to 94° C., then 94° C. for 10 see, followed by 1 degree/sec thermal ramp to 50° C., then 50° C. for 10 sec, followed by 1 degree/sec thermal ramp to 60° C., then 60° C. for 4 minutes.

The cycle sequencing reaction product was cleaned up to remove the unincorporated dye-labeled terminators that can obscure data at the beginning of the sequence. A precipitation protocol was used. To each sequencing reaction in the 96-well plate, 20 μl of Milli-Q water and 60 μl of 100% isopropanol was added. The plate was left at room temperature for at least 20 minutes to precipitate the extension products. The plate was spun in a plate centrifuge (Jouan) at 3,000×g for 30 minutes.

Without disturbing the pellet, the supernatant was discarded by inverting the plate onto several paper tissues (Kimwipes) folded to the size of the plate. The inverted plate, with Kimwipes in place, was placed into the centrifuge (Jouan) and spun at 700×g for 1 minute. The Kimwipes were discarded and the samples were loaded onto a sequencing gel.

Approximately 1 μl of sequencing product was loaded into each well of a 96-lane 5% Long Ranger (FMC single pack) gel. The running buffer consisted of 1×TBE. The glass plates consisted of ABI 48-cm plates for use with a 96-lane 0.4 nun Mylar sharktooth comb. A semi-automated ABI Prism 377-96 DNA sequencer was used (ABI 377 with 96-lane, Big Dye upgrades). Sequencing run settings were as follows: run module 48E-1200, 8 hr collection time, 2400 V electrophoresis voltage, 50 mA electrophoresis current, 200 W electrophoresis power, CCD offset of 0, gel temperature of 51° C., 40 mW laser power, and CCD gain of 2.

The remaining data was generated through pyrosequencing (the data for the “Group II diseases”). Pyrosequencing is a method of sequencing DNA by synthesis, where the addition of one of the four dNTPs that correctly matches the complementary base on the template strand is detected. Detection occurs via utilization of the pyrophosphate molecules liberated upon base addition to the elongating synthetic strand. The pyrophosphate molecules are used to make ATP, which in turn drives the emission of photons in a luciferin/luciferase reaction, and these photons are detected by the instrument.

A Luc96 Pyrosequencer was used under default operating condition supplied by the manufacturer. Primers were designed to anneal within 5 bases of the polymorphism, to serve as sequencing primers. Patient genomic DNA was subject to PCR using amplifying primers that amplify an approximately 200 base pair amplicon containing the polymorphisms of interest. One of the amplifying primers, whose orientation is opposite to the sequencing primer, was biotinylated. This allowed selection of single stranded template for pyrosequencing, whose orientation is complementary to the sequencing primer. Amplicons prepared from genomic DNA were isolated by binding to streptavidin-coated magnetic beads. After denaturation in NaOH, the biotinylated strands were separated from their complementary strands using magnetics. After washing the magnetic beads, the biotinylated template strands still bound to the beads were transferred into 96well plates. The sequencing primers were added, annealing was carried out at 950 for 2 minutes, and plates were placed in the Pyrosequencer. The enzymes, substrates and dNTPs used for synthesis and pyrophosphate detection were added to the instrument immediately prior to sequencing.

The Luc96 software requires definition of a program of adding the four dNTPs that is specific for the location of the sequencing primer, the DNA composition flanking the SNP, and the two possible alleles at the polymorphic locus. This order of adding the bases generates theoretical outcomes of light intensity patterns for each of the two possible homozygous states and the single heterozygous state. The Luc96 software then compares the actual outcome to the theoretical outcome and calls a genotype for each well. Each sample is also assigned one of three confidence scores: pass, uncertain, fail. The results for each plate are output as a text file and processed in Excel using a Visual Basic program to generate a report of genotype and allele frequencies for the various disease and population cell groupings represented on the 96 well plate.

Prediction of potential transcription binding factor sites was performed using a commercially available software program [GENOMATIX MatInspector Professional; URL: http//genomatix.gsfde/cgi-bin/matinspector/matinspector.pl; Quandt et al., Nucleic Acids Res., 23: 4878-4884 (1995)].

### Example 2

A to G Substitution at Position 520 of Human vHL Promotor

TABLE 1
ALLELE FREQUENCY

| | A | G |
|---|---|---|
| CONTROL | | |
| Black men (n = 40 chromosomes) | 13 (33%) | 27 (68%) |
| Black women (n = 38 chromosomes) | 10 (26%) | 28 (74%) |
| White men (n = 34 chromosomes) | 30 (88%) | 4 (12%) |
| White women (n = 40 chromosomes) | 27 (68%) | 13 (33%) |
| DISEASE | | |
| HYPERTENSION | | |
| Black men (n = 16 chromosomes) | 3 (19%) | 13 (81%) |
| Black women (n = 22 chromosomes) | 10 (45%) | 12 (55%) |
| White men (n = 24 chromosomes) | 20 (83%) | 4 (17%) |
| White women n = 16 chromosomes | 10 (63%) | 6 (38%) |
| ESRD due to HYPERTENSION | | |
| Black men (n = 20 chromosomes) | 2 (10%) | 18 (90%) |
| Black women (n = 20 chromosomes) | 6 (30%) | 14 (70%) |
| White men (n = 16 chromosomes) | 10 (63%) | 6 (38%) |
| White women (n = 14 chromosomes) | 12 (86%) | 2 (14%) |
| NIDDM | | |
| Black men (n = 12 chromosomes) | 0 (0%) | 12 (100%) |
| Black women (n = 20 chromosomes) | 4 (20%) | 16 (80%) |
| White men (n = 18 chromosomes) | 10 (56%) | 8 (44%) |
| White women (n = 20 chromosomes) | 12 (60%) | 8 (40%) |
| ESRD due to NIDDM | | |
| Black men (n = 14 chromosomes) | 1 (7%) | 13 (93%) |
| Black women (n = 22 chromosomes) | 6 (27%) | 16 (73%) |
| White men (n = 16 chromosomes) | 8 (50%) | 8 (50%) |
| White women (n = 12 chromosomes) | 6 (50%) | 6 (50%) |

TABLE 2
GENOTYPE FREQUENCIES

| | A/A | A/G | G/G |
|---|---|---|---|
| CONTROLS | | | |
| Black men (n = 20) | 3 (15%) | 7 (35%) | 10 (50%) |
| Black women (n = 19) | 2 (11%) | 6 (32%) | 11 (58%) |
| White men (n = 17) | 14 (82%) | 2 (12%) | 1 (6%) |
| White women (n = 20) | 11 (55%) | 5 (25%) | 4 (20%) |
| DISEASE | | | |
| HYPERTENSION | | | |
| Black men (n = 8) | 0 (0%) | 3 (38%) | 5 (63%) |
| Black women (n = 11) | 3 (27%) | 4 (36%) | 4 (36%) |
| White men (n = 12) | 9 (75%) | 2 (17%) | 1 (8%) |
| White women (n = 8) | 4 (50%) | 2 (25%) | 2 (25%) |
| ESRD due to HYPERTENSION | | | |
| Black men (n = 40) | 0 (0%) | 2 (20%) | 8 (80%) |
| Black women (n = 10) | 2 (20%) | 2 (20%) | 6 (60%) |
| White men (n = 8) | 4 (50%) | 2 (25%) | 2 (25%) |
| White women (n = 7) | 5 (71%) | 2 (29%) | 0 (0%) |
| NIDDM | | | |
| Black men (n = 6) | 0 (0%) | 0 (0%) | 6 (100%) |
| Black women (n = 10) | 1 (10%) | 2 (20%) | 7 (70%) |
| White men (n = 9) | 4 (44%) | 2 (22%) | 3 (33%) |
| White women (n = 10) | 5 (50%) | 2 (20%) | 3 (30%) |
| ESRD due to NIDDM | | | |
| Black men (n = 7) | 0 (0%) | 1 (14%) | 6 (86%) |
| Black women (n = 11) | 2 (18%) | 2 (18%) | 7 (64%) |
| White men (n = 8) | 3 (38%) | 2 (25%) | 3 (38%) |
| White women (n = 6) | 2 (33%) | 2 (33%) | 2 (33%) |

Allele-Specific Odds Ratios

The susceptibility allele is indicated below, as well as the odds ratio (OR). The allele which is present more often in the given disease category was chosen as the susceptibility allele. Haldane's correction was used if the denominator was zero (multiplying all cells by 2 and adding 1). An odds ratio incorporating Haldane's correction is indicated by a superscript “H.” If the odds ratio (OR) was ≧1.5, the 95% confidence interval (C.I.) is also given. An odds ratio of 1.5 was chosen as the threshold of significance based on the recommendation of Austin et al., in Epidemiol. Rev., 16:65-76, 1994. “[E]pidemiology in general and case-control studies in particular are not well suited for detecting weak associations (odds ratios<1.5).” Id. at 66.

An example of the allele-specific odds ratio calculation is given below:

Hypertension: White women

| | Cases | Controls |
|---|---|---|
| G | 6 | 13 |
| A | 10 | 27 |

The odds ratio is (6)(27)/(10)(13)=1.2. Therefore, white women with the G allele have a 1.2 fold higher risk of developing hypertension than white women without the G allele.

If one of the cells contained a 0 which made the denominator in the odds ratio calculation a 0, Haldane's correction was employed. An example of that calculation follows:

NDDM: Black Men

| | Cases | Control |
|---|---|---|
| G | 12 | 27 |
| A | 0 | 13 |

Using Haldane's correction (multiplying all cells by 2 and adding 1), this 2×2 table becomes:

NIDDM: Black Men

| | Cases | Controls |
|---|---|---|
| G | 25 | 55 |
| A | 1 | 27 |

The odds ratio is (25)(27)/(1)(55)=12.3. Therefore, black men with the G allele have 12.3 fold higher risk of developing NIDDM than those black men without the G allele. Odds ratios of 1.5 or greater are highlighted below.

TABLE 3
ALLELE-SPECIFIC ODDS RATIOS

| | SUSCEPTIBILITY | | |
|---|---|---|---|
| DISEASE | ALLELE | OR | 95% C.I. |
| HYPERTENSION | | | |
| Black men | G | 2.1 | 0.5-8.6 |
| Black women | A | 2.3 | 0.8-7.1 |
| White men | G | 1.5 | 0.3-6.7 |
| White women | G | 1.2 | |
| ESRD due to HTN* | | | |
| Black men | G | 2.1 | 0.3-14.3 |
| Black women | G | 1.9 | 0.5-6.9 |
| White men | G | 3.0 | 0.7-13.1 |
| White women | A | 3.6 | 0.6-14.8 |
| NIDDM | | | |
| Black men | G | 12.3^{H} | 1.6-95.4 |
| Black women | G | 1.4 | |
| White men | G | 6.0 | 1.5-24.3 |
| White women | G | 1.4 | |
| ESRD due to NIDDM*¹ | | | |
| Black men | A | 2.8^{H} | 0.3-28.5 |
| Black women | A | 1.5 | 0.4-6.3 |
| White men | G | 1.3 | |
| White women | G | 1.5 | 0.4-6.3 |

*Compared to HTN alone.
*¹Compared to NIDDM alone.

Genotype-Specific Odds Ratios

The susceptibility allele (S) is indicated; the alternative allele at this locus is defined as the protective allele (P). Also presented are the odds ratio (OR) for the SS and SP genotypes; the odds ratio for the PP genotype is 1, since it is the reference group, and is not presented separately. For odds ratios>1.5, the 95% confidence interval (C.I.) is also given, in parentheses. An odds ratio of 1.5 was chosen as the threshold of significance based on the recommendation of Austin et al., in Epidemiol. Rev., 16:65-76, 1994. [E]pidemiology in general and case-control studies in particular are not well suited for detecting weak associations (odds ratios <1.5).” Id. at 66.

Where Haldane's zero cell correction was employed, the odds ratio is so indicated with a superscript “H,”. To minimize confusion, genotype-specific odds ratios are presented only for diseases in which the allele-specific odds ratio was at least 1.5.

An example is worked below, assuming that G is the susceptibility allele (S), and A is the protective allele (P).

Black men: Hypertension

| | Cases | Controls |
|---|---|---|
| GG (SS) | 5 | 10 |
| GA (SP) | 3 | 7 |
| AA (PP) | 0 | 3 |

Applying Haldane's correction, the above 2×3 table becomes:

Black men: Hypertension

| | Cases | Controls | Odds Ratio |
|---|---|---|---|
| GG (SS) | 11 | 21 | (11) (7)/(21) (1) = 3.7 |
| GA (SP) | 7 | 15 | (7) (7)/(15) (1) = 3.3 |
| AA (PP) | 1 | 7 | 1.0 (by definition) |

The odds ratios for individual genotypes are given below. Odds ratios of 1.5 or more are highlighted.

TABLE 4
GENOTYPE-SPECIFIC ODDS RATIOS

| | SUSCEP- | | |
|---|---|---|---|
| | TIBILITY | | |
| DISEASE | ALLELE | OR (SS) | OR (SP) |
| HYPERTENSION | | | |
| Black men | G | 3.7 (0.4-33.7)^{H} | 3.3 (0.3-31.9)^{H} |
| Black women | A | 4.1 (0.5-34.5) | 1.8 (0.3-10.1) |
| White men | G | 1.6 (0.1-28.1) | 1.6 (0.2-13.1) |
| ESRD due to HTN* | | | |
| Black men | G | 5.7 (0.6-50.7)^{H} | 2.3 (0.2-23.9)^{H} |
| Black women | G | 2.3 (0.3-20.1) | 0.8 |
| White men | G | 4.5 (0.3-65.2) | 2.3 (0.2-22.1) |
| White women | A | 4.3 (0.5-38.4)^{H} | 4.1 (0.4-41.7)^{H} |
| NIDDM | | | |
| Black men | G | 4.3 (0.5-39.4)^{H} | 0.5 (0-8.6)^{H} |
| White men | G | 10.5 (0.8-130.7) | 3.5 (0.4-33.3) |
| ESRD due to NIDDM*¹ | | | |
| Black men | A | 1.0^{H} | 3.0 (0.3-33)^{H} |
| Black women | A | 2.0 (0.1-27.4) | 1.0 |
| White women | G | 1.7 (0.1-18.9) | 2.5 (0.2-32.2) |

*Compared to HTN alone.
*¹Compared to NIDDM alone.

PCR and sequencing were conducted as described in Example 1. The primers used were those in Example 1. The control samples were in good agreement with Hardy-Weinberg equilibrium, as follows:

A frequency of 0.33 for the A allele (“p”) and 0.68 for the G allele (“q”) among black male control individuals predicts genotype frequencies of 11% A/A, 43% A/G, and 46% GIG at Hardy-Weinberg equilibrium (2+2pq+q²=1). The observed genotype frequencies were 15% A/A, 35% A/G, and 50% G/G, in fair agreement with those predicted for Hardy-Weinberg equilibrium.

A frequency of 0.26 for the A allele (“p”) and 0.74 for the G allele (“q”) among black female control individuals predicts genotype frequencies of 7% A/A, 38% A/G, and 55% GIG at Hardy-Weinberg equilibrium (p²+2pq+q²=1). The observed genotype frequencies were 11% A/A, 32% A/G, and 58% G/G, in close agreement with those predicted for Hardy-Weinberg equilibrium.

A frequency of 0.88 for the A allele (“p”) and 0.12 for the G allele (“q”) among white male control individuals predicts genotype frequencies of 77% A/A, 22% A/G, and 1% G/G at Hardy-Weinberg equilibrium (p²+2pq+q²=1). The observed genotype frequencies were 82% A/A, 12% A/G, and 6% G/G, in fair agreement with those predicted for Hardy-Weinberg equilibrium.

A frequency of 0.68 for the A allele (“p”) and 0.33 for the G allele (“q”) among white female control individuals predicts genotype frequencies of 46% A/A, 43% A/G, and 11% G/G at Hardy-Weinberg equilibrium (p²+2pq+q² 1). The observed genotype frequencies were 55% A/A, 25% A/G, and 20% G/G, in distant agreement with those predicted for Hardy-Weinberg equilibrium.

Using an allele-specific odds ratio of 1.5 or greater as a practical level of significance (see Austin et al., discussed above), the following observations can be made.

For black men with hypertension, the odds ratio for the G allele as a risk factor for disease was 2.1 (95% CI, 0.5-8.6). The odds ratio for the homozygote (GG) was 3.7 (95% CI, 0.4-33.7)^{H}. The heterozygote (AG genotype) had a similar odds ratio of 3.3 (95% C.I., 0.3-31.9)^{H}. These data suggest that the G allele behaves as a classical dominant allele, since the heterozygote had essentially the same odds ratio as the homozygote.

For black women with hypertension, the odds ratio for the A allele as a risk factor for disease was 2.3 (95% CI, 0.8-7.1). The odds ratio for the homozygote (AA) was 4.1 (95% Cl, 0.5-34.5), whereas the heterozygote (AG genotype) had an odds ratio of only 1.8 (95% CI, 0.3-10.1). These data suggest that the A allele behaves as a dominant allele, with a more than multiplicative effect of allele dosage [4.1>(1.8)(1.8)=3.2].

For white men with hypertension, the odds ratio for the G allele as a risk factor for disease was 1.5 (95% CI, 0.3-6.7). The odds ratio for the homozygote (GG) was 1.6 (95% CI, 0.1-28.1). The heterozygote (GA genotype) had the same odds ratio of 1.6 (95% C.I., 0.2-13.1). These data suggest that the G allele behaves as a classical dominant allele, since the heterozygote had the same odds ratio as the homozygote.

For black men with end-stage renal disease ESRD due to HTN, the odds ratio for the G allele as a risk factor for disease was 2.1 (95% CI, 0.3-14.3) relative to black men with hypertension but no renal disease. The odds ratio for the homozygote (GG) was 5.7 (95% CI, 0.6-50.7)^{H}, whereas the heterozygote (GA genotype) had an odds ratio of 2.3 (95% CI, 0.2-23.9)^{H}. These data suggest that the G allele behaves as a dominant allele, with a multiplicative effect of allele dosage [5.7˜(2.3)(2.3)=5.3].

For black women with ESRD due to HTN, the odds ratio for the G allele as a risk factor for disease was 1.9 (95% CI, 0.5-6.9) relative to black women with hypertension but no renal disease. The odds ratio for the homozygote (GG) was 2.3 (95% CI, 0.3-20.1), while the heterozygote (GA genotype) had an odds ratio close to 1 (0.8). These data suggest that the G allele behaves in a recessive fashion.

For white men with ESRD due to HTN, the odds ratio for the G allele as a risk factor for disease was 3.0 (95% Cl, 0.7-13.1) relative to white men with hypertension but no renal disease. The odds ratio for the homozygote (GG) was 4.5 (95% CI, 0.3-65.2), whereas the heterozygote (GA genotype) had an odds ratio of 2.3 (95% CI, 0.2-22.1). These data suggest that the G allele behaves as a dominant allele, with more than an additive effect of allele dosage [4.5>2.3+2.3−1=3.6].

For white women with ESRD due to HTN, the odds ratio for the A allele as a risk factor for disease was 2.9 (95% CI, 0.6-14.8) relative to white women with hypertension but no renal disease. The odds ratio for the homozygote (AA) was 4.3 (95% Cl, 0.53.4)^{H}. The heterozygote (AG genotype) had essentially the same odds ratio of 4.1 (95% C.I., 0.4-41.7)^{H}. These data suggest that the A allele behaves as a classical dominant allele, since the heterozygote had the same odds ratio as the homozygote.

For black men with NIDDM, the odds ratio for the G allele as a risk factor for disease was 12.3 (95% CI, 1.6-95.4)^{H}. The odds ratio for the homozygote (GG) was 4.3 (95% CI, 0.5-39.4)^{H}, while the heterozygote (GA genotype) actually had an odds ratio indistinguishable from 1 [0.5 (95% CI, 0-8.6)^{H}]. These data suggest that the G allele behaves in a recessive fashion.

For white men with NIDDM, the odds ratio for the G allele as a risk factor for disease was 6.0 (95% Cl, 1.5-24.3). The odds ratio for the homozygote (GG) was 10.5 (95% CI, 0.8-130.7), whereas the heterozygote (GA genotype) had an odds ratio of 3.5 (95% CI, 0.4-33.3). These data suggest that the G allele behaves as a dominant allele, with a less than multiplicative effect of allele dosage [10.5<(3.5)(3.5)=12.3].

For black men with ESRD due to NIDDM, the odds ratio for the A allele as a risk factor for disease was 2.8 (95% Cr, 0.3-2.5)^{H} relative to black men with NIDDM but no renal disease. The odds ratio for the homozygote (AA) was 1.0^{H}, whereas the heterozygote (AG genotype) had a higher odds ratio of 3.0 (95% C.I., 0.3-33)^{H}. These data suggest that the A allele behaves in a co-dominant manner.

For black women with ESRD due to NIDDM, the odds ratio for the A allele as a risk factor for disease was 1.5 (95% CI, 0.4-6.3) relative to black women with NIDDM but no renal disease. The odds ratio for the homozygote (AA) was 2.0 (95% CI, 0.1-27.4), whereas the heterozygote (AG genotype) had an odds ratio of 1.0. These data suggest that the A allele behaves in a classical recessive manner.

For white women with ESRD due to NIDDK, the odds ratio for the G allele as a risk factor for disease was 1.5 (95% CI, 0.4-6.3) relative to white women with NIDDM but no renal disease. The odds ratio for the homozygote (GG) was 1.7 (95% CI, 0.1-18.9), while the heterozygote (GA genotype) had a similar odds ratio of 2.5 (95% CI, 0.2-32.2). These data suggest that the G allele behaves in a dominant fashion.

According to GENOMATIX MatInspector, the A520->G SNP potentially disrupts several important transcription factor binding sites, as follows:

a. A potential binding site for hepatic leukemia factor (HLF_{—}01 as abbreviated by GENOMATIX; Hunger et al., Mol. Cell Biol., 14:5986-5996, 1994). The consensus binding sequence for HLF 01 consists of the 10 nucleotides 5′RTTACRYAAT-3′ (SEQ ID NO: 4). This sequence occurs between nucleotides 512 and 521, inclusive, on the (+) strand, with a matrix score of 0.845 (where 1.000 represents a perfect match). The A520->G SNP replaces the indicated A with a G. HLF_{—}01 binding sites occur on average 1.69 times per 1000 base pairs of random vertebrate genomic DNA.

b. A potential binding site for the PAR-type chicken vitellogenin promoter-b (VBPF, for chicken vitellogenin gene binding protein factor; Haas et al., Mol. Cell Biol., 15:1923-1932, 1995). The consensus binding sequence for VBPF consists of the ten nucleotides 5′-GTTACRTNAN-3′. This sequence occurs between nucleotides 512 and 521, inclusive, on the (+) strand, with a matrix score of 0.884 (where 1.000 represents a perfect match). The A520->G SN-P replaces the indicated A with a G. VBPF binding sites occur on average 3.78 times per 1000 base pairs of random vertebrate genomic DNA.

c. A potential binding site for the CCAAT/Enhancer Binding Protein (CEBP_C). The consensus binding sequence for CEBP_C consists of the complement to the following 18 nucleotides 5′-TRTNNMTTRCMNMAVCN-3′ (SEQ ID NO: 5) (nucleotides 507-524). Their complement is located on the (−) strand. The match for this sequence has a score of 0.855, where 1.000 represents a perfect match. The A520->G SNP replaces the indicated A with a G. CEBP_C binding sites occur rarely, on average 0.27 times per 1000 base pairs of random vertebrate genonic DNA.

d. A potential binding site for the SL3-3 enhancer factor 1 (abbreviated SEF1_C by GENOMATIX). SEF1 denotes a family of proteins which bind to a T cell-specific enhancer of the SL3-3 mouse leukemia virus, as well as to similar enhancers in cellular genes, including the T cell antigen receptor (Hallberg et al., Nucl. Acids Res., 20(24):6495-6499, 1992; Thornell, J. Biol. Chem., 268(29):21946-21954, 1993). The consensus binding sequence for SEF 1 consists of the 19 nucleotides complementary to 5′RACCACAGATATCCNTGTT-3′ (SEQ ID NO: 6). The complement is located on the (−) strand, across from nucleotides 514-532 on the (+) strand. The match for this sequence has a score of 0.712, where 1.000 represents a perfect match. The A520->G SNTP replaces the indicated A with a G. SEF1_C binding sites occur extremely rarely, less than 0.01 times per 1000 base pairs of random vertebrate genomic DNA.

### Example 3

### C to T Substitution at Position 638 of Human vHL Promoter

TABLE 5
ALLELE FREQUENCY

| | C | T |
|---|---|---|
| CONTROL | | |
| Black men (n = 38 chromosomes) | 32 (84%) | 6 (16%) |
| Black women (n = 38 chromosomes) | 35 (92%) | 3 (8%) |
| White men (n = 34 chromosomes) | 34 (100%) | 0 (0%) |
| White women (n = 42 chromosomes) | 42 (100%) | 0 (0%) |
| DISEASE | | |
| HYPERTENSION | | |
| Black men (n = 20 chromosomes) | 18 (90%) | 2 (10%) |
| Black women (n = 22 chromosomes) | 19 (86%) | 3 (14%) |
| White men (n = 24 chromosomes) | 24 (100%) | 0 (0%) |
| Whitc women (n = 16 chromosomes) | 16 (100%) | 0 (0%) |
| ESRD due to HYPERTENSION | | |
| Black men (n = 20 chromosomes) | 19 (95%) | 1 (5%) |
| Black women (n = 18 chromosomes) | 16 (89%) | 2 (11%) |
| White men (n = 18 chromosomes) | 18 (100%) | 0 (0%) |
| White women (n = 20 chromosomes) | 20 (100%) | 0 (0%) |
| NTDDM | | |
| Black men (n = 8 chromosomes) | 5 (63%) | 3 (38%) |
| Black women (n = 14 chromosomes) | 12 (86%) | 2 (14%) |
| White men (n = 16 chromosomes) | 16 (100%) | 0 (0%) |
| White women (n = 14 chromosomes) | 13 (93%) | 1 (7%) |
| ESRD due to NIDDM | | |
| Black men (n = 10 chromosomes) | 10 (100%) | 0 (0%) |
| Black women (n = 14 chromosomes) | 12 (86%) | 2 (14%) |
| White men (n = 12 chromosomes) | 12 (100%) | 0 (0%) |
| White women (n = 10 chromosomes) | 10 (100%) | 0 (0%) |

TABLE 6
ALLELE FREQUENCY

| Disease | Race | CHROMOSOMES | N | C | N | T |
|---|---|---|---|---|---|---|
| Controls | African-American | 90 | 75 | 83.3% | 15 | 16.7% |
| | Caucasian | 88 | 88 | 100.0% | 0 | 0.0% |
| Colon cancer | African-American | 48 | 41 | 85.4% | 7 | 14.6% |
| | Caucasian | 46 | 46 | 100.0% | 0 | 0.0% |
| Hypertension | African-American | 44 | 40 | 90.9% | 4 | 9.1% |
| | Caucasian | 44 | 44 | 100.0% | 0 | 0.0% |
| ASPVD due to HTN | African-American | 52 | 47 | 90.4% | 5 | 9.6% |
| | Caucasian | 50 | 50 | 100.0% | 0 | 0.0% |
| CVA due to HTN | African-American | 48 | 33 | 68.8% | 15 | 31.3% |
| | Caucasian | 44 | 44 | 100.0% | 0 | 0.0% |
| Cataracts due to HTN | African-American | 44 | 41 | 93.2% | 3 | 6.8% |
| | Caucasian | 42 | 41 | 97.6% | 1 | 2.4% |
| HTN CM | African-American | 48 | 45 | 93.8% | 3 | 6.3% |
| | Caucasian | 46 | 46 | 100.0% | 0 | 0.0% |
| MI due to HTN | African-American | 40 | 32 | 80.0% | 8 | 20.0% |
| | Caucasian | 44 | 44 | 100.0% | 0 | 0.0% |
| NIDDM | African-American | 44 | 35 | 79.5% | 9 | 20.5% |
| | Caucasian | 46 | 46 | 100.0% | 0 | 0.0% |
| ASPVD due to NIDDM | African-American | 46 | 39 | 84.8% | 7 | 15.2% |
| | Caucasian | 46 | 46 | 100.0% | 0 | 0.0% |
| CVA due to NIDDM | African-American | 48 | 39 | 81.3% | 9 | 18.8% |
| | Caucasian | 44 | 44 | 100.0% | 0 | 0.0% |
| Ischemic CM | African-American | 48 | 41 | 85.4% | 7 | 14.6% |
| | Caucasian | 42 | 42 | 100.0% | 0 | 0.0% |
| Ischemic CM with NIDDM | African-American | 44 | 37 | 84.1% | 7 | 15.9% |
| | Caucasian | 46 | 46 | 100.0% | 0 | 0.0% |
| MI due to NIDDM | African-American | 46 | 43 | 93.5% | 3 | 6.5% |
| | Caucasian | 48 | 48 | 100.0% | 0 | 0.0% |
| Afib without valvular disease | African-American | 46 | 40 | 87.0% | 6 | 13.0% |
| | Caucasian | 48 | 48 | 100.0% | 0 | 0.0% |
| Alcohol abuse | African-American | 48 | 40 | 83.3% | 8 | 16.7% |
| | Caucasian | 48 | 48 | 100.0% | 0 | 0.0% |
| Alcoholic cirrhosis | African-American | 48 | 39 | 81.3% | 9 | 18.8% |
| Anxiety | African-American | 44 | 34 | 77.3% | 10 | 22.7% |
| | Caucasian | 42 | 42 | 100.0% | 0 | 0.0% |
| Asthma | African-American | 48 | 48 | 100.0% | 0 | 0.0% |
| | Caucasian | 46 | 46 | 100.0% | 0 | 0.0% |
| COPD | African-American | 40 | 35 | 87.5% | 5 | 12.5% |
| | Caucasian | 42 | 40 | 95.2% | 2 | 4.8% |
| Cholecystectomy | African-American | 48 | 38 | 79.2% | 10 | 20.8% |
| | Caucasian | 44 | 44 | 100.0% | 0 | 0.0% |
| DJD | African-American | 40 | 35 | 87.5% | 5 | 12.5% |
| | Caucasian | 40 | 40 | 100.0% | 0 | 0.0% |
| ESRD and frequent de-clots | African-American | 46 | 40 | 87.0% | 6 | 13.0% |
| | Caucasian | 40 | 40 | 100.0% | 0 | 0.0% |
| ESRD due to FSGS | African-American | 44 | 29 | 65.9% | 15 | 34.1% |
| | Caucasian | 44 | 44 | 100.0% | 0 | 0.0% |
| ESRD due to IDDM | African-American | 48 | 33 | 68.8% | 15 | 31.3% |
| Seizure disorder | African-American | 48 | 39 | 81.3% | 9 | 18.8% |
| | Caucasian | 48 | 48 | 100.0% | 0 | 0.0% |

TABLE 7
GENOTYPE FREQUENCIES

| | C/C | C/T | T/T |
|---|---|---|---|
| CONTROLS | | | |
| Black men (n = 19) | 15 (79%) | 2 (11%) | 2 (11%) |
| Black women (n = 19) | 16 (84%) | 3 (16%) | 0 (0%) |
| White men (n = 17) | 17 (100%) | 0 (0%) | 0 (0%) |
| White women (n = 21) | 21 (100%) | 0 (0%) | 0 (0%) |
| DISEASE | | | |
| HYPERTENSION | | | |
| Black men (n = 10) | 8 (80%) | 2 (20%) | 0 (0%) |
| Black women (n = 11) | 8 (73%) | 3 (27%) | 0 (0%) |
| White men (n = 12) | 12 (100%) | 0 (0%) | 0 (0%) |
| White women (n = 8) | 8 (100%) | 0 (0%) | 0 (0%) |
| ESRD due to | | | |
| HYPERTENSION | | | |
| Black men (n = 10) | 9 (90%) | 1 (10%) | 0 (0%) |
| Black women (n = 9) | 7 (78%) | 2 (22%) | 0 (0%) |
| White men (n = 9) | 9 (100%) | 0 (0%) | 0 (0%) |
| White women (n = 10) | 10 (100%) | 0 (0%) | 0 (0%) |
| NIDDM | | | |
| Black men (n = 4) | 1 (25%) | 3 (75%) | 0 (0%) |
| Black women (n = 7) | 5 (71%) | 2 (29%) | 0 (0%) |
| White men (n = 8) | 8 (100%) | 0 (0%) | 0 (0%) |
| White women (n = 7) | 6 (86%) | 1 (14%) | 0 (0%) |
| ESRD due to NIDDM | | | |
| Black men (n = 5) | 5 (100%) | 0 (0%) | 0 (0%) |
| Black women (n = 7) | 5 (71%) | 2 (29%) | 0 (0%) |
| White men (n = 6) | 6 (100%) | 0 (0%) | 0 (0%) |
| White women (n = 5) | 5 (100%) | 0 (0%) | 0 (0%) |

TABLE 8
GENOTYPE FREQUENCIES

| Disease | Race | People | N | C/C | N | C/T | N | T/T |
|---|---|---|---|---|---|---|---|---|
| Controls | African-American | 45 | 37 | 82.2% | 1 | 2.2% | 7 | 15.6% |
| | Caucasian | 44 | 44 | 100.0% | 0 | 0.0% | 0 | 0.0% |
| Colon cancer | African-American | 24 | 20 | 83.3% | 1 | 4.2% | 3 | 12.5% |
| | Caucasian | 23 | 23 | 100.0% | 0 | 0.0% | 0 | 0.0% |
| Hypertension | African-American | 22 | 18 | 81.8% | 4 | 18.2% | 0 | 0.0% |
| | Caucasian | 22 | 22 | 100.0% | 0 | 0.0% | 0 | 0.0% |
| ASPVD due to HTN | African-American | 26 | 23 | 88.5% | 1 | 3.8% | 2 | 7.7% |
| | Caucasian | 25 | 25 | 100.0% | 0 | 0.0% | 0 | 0.0% |
| CVA due to HTN | African-American | 24 | 15 | 62.5% | 3 | 12.5% | 6 | 25.0% |
| | Caucasian | 22 | 22 | 100.0% | 0 | 0.0% | 0 | 0.0% |
| Cataracts due to HTN | African-American | 22 | 19 | 86.4% | 3 | 13.6% | 0 | 0.0% |
| | Caucasian | 21 | 20 | 95.2% | 1 | 4.8% | 0 | 0.0% |
| HTN CM | African-American | 24 | 22 | 91.7% | 1 | 4.2% | 1 | 4.2% |
| | Caucasian | 23 | 23 | 100.0% | 0 | 0.0% | 0 | 0.0% |
| MI due to HTN | African-American | 20 | 14 | 70.0% | 4 | 20.0% | 2 | 10.0% |
| | Caucasian | 22 | 22 | 100.0% | 0 | 0.0% | 0 | 0.0% |
| NIDDM | African-American | 22 | 16 | 72.7% | 3 | 13.6% | 3 | 13.6% |
| | Caucasian | 23 | 23 | 100.0% | 0 | 0.0% | 0 | 0.0% |
| ASPVD due to NIDDM | African-American | 23 | 19 | 82.6% | 1 | 4.3% | 3 | 13.0% |
| | Caucasian | 23 | 23 | 100.0% | 0 | 0.0% | 0 | 0.0% |
| CVA due to NIDDM | African-American | 24 | 19 | 79.2% | 1 | 4.2% | 4 | 16.7% |
| | Caucasian | 22 | 22 | 100.0% | 0 | 0.0% | 0 | 0.0% |
| Ischemic CM | African-American | 24 | 18 | 75.0% | 5 | 20.8% | 1 | 4.2% |
| | Caucasian | 21 | 21 | 100.0% | 0 | 0.0% | 0 | 0.0% |
| Ischemic CM with NIDDM | African-American | 22 | 17 | 77.3% | 3 | 13.6% | 2 | 9.1% |
| | Caucasian | 23 | 23 | 100.0% | 0 | 0.0% | 0 | 0.0% |
| MI due to NIDDM | African-American | 23 | 21 | 91.3% | 1 | 4.3% | 1 | 4.3% |
| | Caucasian | 24 | 24 | 100.0% | 0 | 0.0% | 0 | 0.0% |
| Afib without valvular disease | African-American | 23 | 20 | 87.0% | 0 | 0.0% | 3 | 13.0% |
| | Caucasian | 24 | 24 | 100.0% | 0 | 0.0% | 0 | 0.0% |
| Alcohol abuse | African-American | 24 | 20 | 83.3% | 0 | 0.0% | 4 | 16.7% |
| | Caucasian | 24 | 24 | 100.0% | 0 | 0.0% | 0 | 0.0% |
| Alcoholic cirrhosis | African-American | 24 | 19 | 79.2% | 1 | 4.2% | 4 | 16.7% |
| Anxiety | African-American | 22 | 17 | 77.3% | 0 | 0.0% | 5 | 22.7% |
| | Caucasian | 21 | 21 | 100.0% | 0 | 0.0% | 0 | 0.0% |
| Asthma | African-American | 24 | 24 | 100.0% | 0 | 0.0% | 0 | 0.0% |
| | Caucasian | 23 | 23 | 100.0% | 0 | 0.0% | 0 | 0.0% |
| COPD | African-American | 20 | 16 | 80.0% | 3 | 15.0% | 1 | 5.0% |
| | Caucasian | 21 | 20 | 95.2% | 0 | 0.0% | 1 | 4.8% |
| Cholecystectomy | African-American | 24 | 19 | 79.2% | 0 | 0.0% | 5 | 20.8% |
| | Caucasian | 22 | 22 | 100.0% | 0 | 0.0% | 0 | 0.0% |
| DID | African-American | 20 | 17 | 85.0% | 1 | 5.0% | 2 | 10.0% |
| | Caucasian | 20 | 20 | 100.0% | 0 | 0.0% | 0 | 0.0% |
| ESRD and frequent de-clots | African-American | 23 | 19 | 82.6% | 2 | 8.7% | 2 | 8.7% |
| | Caucasian | 20 | 20 | 100.0% | 0 | 0.0% | 0 | 0.0% |
| ESRD due to FSGS | African-American | 22 | 13 | 59.1% | 3 | 13.6% | 6 | 27.3% |
| | Caucasian | 22 | 22 | 100.0% | 0 | 0.0% | 0 | 0.0% |
| ESRD due to LDDM | African-American | 24 | 16 | 66.7% | 1 | 4.2% | 7 | 29.2% |
| Seizure disorder | African-American | 24 | 18 | 75.0% | 3 | 12.5% | 3 | 12.5% |
| | Caucasian | 24 | 24 | 100.0% | 0 | 0.0% | 0 | 0.0% |

Allele-Specific Odds Ratios

The susceptibility allele is indicated below, as well as the odds ratio (OR). The allele which is present more often in the given disease category was chosen as the susceptibility allele. Haldane's correction was used if the denominator was zero (multiplying all cells by 2 and adding 1). An odds ratio incorporating Haldane's correction is indicated by a superscript “H.” If the odds ratio (OR) was ≧1.5, the 95% confidence interval (C.I.) is also given. An odds ratio of 1.5 was chosen as the threshold of significance based on the recommendation of Austin et al., in Epidemiol. Rev., 16:6576, 1994. “[E]pidemiology in general and case-control studies in particular are not well suited for detecting weak associations (odds ratios <1.5).” Id. at 66.

TABLE 9
ALLELE-SPECIFIC ODDS RATIOS

| | SUSCEPTIBILITY | | |
|---|---|---|---|
| DISEASE | ALLELE | OR | 95% C.I. |
| Hypertension | | | |
| Black men | C | 1.7 | 0.3-9.2 |
| Black women | T | 1.8 | 0.3-10.0 |
| White men | C | 1.0 | |
| White women | C | 1.0 | |
| ESRD due to HTN* | | | |
| Black men | C | 2.1 | 0.2-25.3 |
| Black women | C | 1.3 | |
| White men | C | 1.0 | |
| White women | C | 1.0 | |
| NIDDM | | | |
| Black men | T | 3.2 | 0.6-17.1 |
| Black women | T | 1.9 | 0.3-13.1 |
| White men | C | 1.0 | |
| White women | T | 9.4^{H} | 0.9-94.6 |
| ESRD due to NIDDM*¹ | | | |
| Black men | C | 13.4^{H} | 1.5-123 |
| Black women | C | 1.0 | |
| White men | C | 1.0 | |
| White women | C | 2.3^{H} | 0.2-24.1 |

*Compared to HTN alone.
*¹Compared to NIDDM alone.

TABLE 10
ALLELE SPECIFIC ODDS RATIOS

| | | | | Lower | Upper | |
|---|---|---|---|---|---|---|
| | | | | Limit | Limit | |
| | | Risk | Odds | 95% | 95% | |
| Disease | Race | Allele | Ratio | CI | CI | Haldane |
| Colon cancer | African American | C | 1.2 | 0.4 | 3.1 | |
| | Caucasian | C | 1.0 | — | — | |
| ASPVD due to HTN* | African American | T | 1.1 | 0.3 | 4.2 | |
| | Caucasian | C | 1.0 | — | — | |
| CVA due to HTN* | African-American | T | 4.5 | 1.4 | 15.0 | |
| | Caucasian | C | 1.0 | — | — | |
| Cataracts due to HTN* | African-American | C | 2.7 | 0.7 | 10.0 | |
| | Caucasian | T | 6.4 | 0.3 | 160.4 | H |
| HTN CM*¹ | African-American | C | 3.8 | 0.9 | 15.2 | |
| | Caucasian | C | 1.0 | — | — | |
| MI due to HTN* | African-American | T | 2.5 | 0.7 | 9.1 | |
| | Caucasian | C | 1.0 | — | — | |
| ASPVD due to NIDDM*² | African-American | C | 1.4 | 0.5 | 43 | |
| | Caucasian | C | 1.0 | — | — | |
| CVA due to NIDDM*² | African-American | C | 1.1 | 0.4 | 3.1 | |
| | Caucasian | C | 1.0 | — | — | |
| Ischemic CM with NIDDM*³ | African-American | T | 2.7 | 0.7 | 11.2 | |
| | Caucasian | C | 1.0 | — | — | |
| MT due to NIDDM*² | African-American | C | 3.7 | 0.9 | 14.7 | |
| | Caucasian | C | 1.0 | — | — | |
| Afib without valvular disease | African-American | C | 1.3 | 0.5 | 3.7 | |
| | Caucasian | C | 1.0 | — | — | |
| Alcohol abuse | African-American | T | 1.0 | 0.4 | 2.6 | |
| | Caucasian | C | 1.0 | — | — | |
| Alcoholic cirrhosis*⁴ | African-American | T | 1.2 | 0.4 | 3.3 | |
| Anxiety | African-American | T | 1.5 | 0.6 | 3.6 | |
| | Caucasian | C | 1.0 | — | — | |
| Asthma | African American | C | 19.9 | 1.2 | 340.6 | H |
| | Caucasian | C | 1.0 | — | — | |
| COPD | African American | C | 1.4 | 0.5 | 4.2 | |
| | Caucasian | T | 10.9 | 0.5 | 232.8 | H |
| Cholecystectomy | African-American | T | 1.3 | 0.5 | 3.2 | |
| | Caucasian | C | 1.0 | — | — | |
| DJD | African-American | C | 1.4 | 0.5 | 4.2 | |
| | Caucasian | C | 1.0 | — | — | |
| ESRD and frequent de-clots | African-American | C | 1.3 | 0.5 | 3.7 | |
| | Caucasian | C | 1.0 | — | — | |
| ESRD due to FSGS | African-American | T | 2.6 | 1.1 | 6.0 | |
| | Caucasian | C | 1.0 | — | — | |
| ESRD due to IDDM | African-American | T | 2.3 | 1.0 | 5.2 | |
| Seizure disorder | African-American | T | 1.2 | 0.5 | 2.9 | |
| | Caucasian | C | 1.0 | — | — | |

*Compared to HTN alone.
*¹Compared to MI with HTN.
*²Compared to NIDDM alone.
*³Compared to MI with NIDDM.
*⁴Compared to Alcohol Abuse

Genotype-Specific Odds Ratios

The susceptibility allele (S) is indicated; the alternative allele at this locus is defined as the protective allele (P). Also presented are the odds ratio (OR) for the SS and SP genotypes; the odds ratio for the PP genotype is 1, since it is the reference group, and is not presented separately. For odds ratios ≧1.5, the 95% confidence interval (C.I.) is also given, in parentheses. An odds ratio of 1.5 was chosen as the threshold of significance based on the recommendation of Austin et at., in Epidemiol. Rev., 16:65-76, 1994. “[E]pidemiology in general and case-control studies in particular are not well suited for detecting weak associations (odds ratios <1.5).” Id. at 66.

Where Haldane's zero cell correction was employed, the odds ratio is so indicated with a superscript “H”. To minimize confusion, genotype-specific odds ratios are presented only for diseases in which the allele-specific odds ratio was at least 1.5. The odds ratios for individual genotypes are given below.

TABLE 11
GENOTYPE-SPECIFIC ODDS RATIOS

| | SUSCEP- | | |
|---|---|---|---|
| | TIBILITY | | |
| DISEASE | ALLELE | OR (SS) | OR (SP) |
| Hypertension | | | |
| Black men | C | 2.7 (0.3-25.4)^{H} | 5.0 (0.4-59.7)^{H} |
| Black women | T | 1.9 (0.1-33.0)^{H} | 2.0 (0.3-12.2) |
| ESRD due to HTN* | | | |
| Black men | C | 3.1 (0.3-28.3)^{H} | 3.0 (0.2-39.6)^{H} |
| NIDDM | | | |
| Black men | T | 2.1 (0.2-24.0)^{H} | 22.5 (1.5-335) |
| Black women | T | 3.0 (0.2-52.1)^{H} | 2.1 (0.3-16.6) |
| White women | T | 3.3 (0.2-56.6)^{H} | 9.9 (0.9-104)^{H} |
| ESRD due to NIDDM*¹ | | | |
| Black men | C | 3.7 (0.2-77.6)^{H} | 0.1 (0-4.6)^{H} |
| White women | C | 0.8 (0-15.2)^{H} | 0.3 (0-11.9)^{H} |

*Compared to HTN alone.
*¹Compared to NIDDM alone.

TABLE 12
GENOTYPE-SPECIFIC ODDS RATIOS

| | | RISK | SS | | SP | |
|---|---|---|---|---|---|---|
| Disease | Race | ALLELE | O.R. | HALDANE | O.R. | HALDANE |
| Colon cancer | African-American | C | 1.3 | | 2.3 | |
| | Caucasian | C | 0.5 | H | 1.0 | H |
| Hypertension | African-American | C | 7.4 | H | 45.0 | H |
| | Caucasian | C | 0.5 | H | 1.0 | H |
| ASPVD due to | African-American | T | 0.0 | | 0.0 | |
| HTN* | Caucasian | C | 1.1 | H | 1.0 | H |
| CVA due to HTN* | African-American | T | 0.0 | | 0.0 | |
| | Caucasian | C | 1.0 | H | 1.0 | H |
| Cataracts due to | African-American | C | 7.8 | H | 35.0 | H |
| HTN* | Caucasian | T | 0.5 | H | 3.0 | H |
| HTN CM*¹ | African-American | C | 3.1 | | 0.5 | |
| | Caucasian | C | 1.0 | H | 1.0 | H |
| MI due to HTN* | African-American | T | 0.0 | | 0.0 | |
| | Caucasian | C | 1.0 | H | 1.0 | H |
| ASPVD due to | African-American | C | 1.2 | | 0.3 | |
| NIDDM*² | Caucasian | C | 1.0 | H | 1.0 | H |
| CVA due to | African-American | C | 0.9 | | 0.3 | |
| NIDDM*² | Caucasian | C | 1.0 | H | 1.0 | H |
| Ischemic CM with | African-American | T | 0.4 | | 1.5 | |
| NIDDM*³ | Caucasian | C | 1.0 | H | 1.0 | H |
| MI due to NIDDM*³ | African-American | C | 3.9 | | 1.0 | |
| | Caucasian | C | 1.0 | H | 1.0 | H |
| Afib without | African-American | C | 1.3 | | 0.0 | |
| valvular disease | Caucasian | C | 0.6 | H | 1.0 | H |
| Alcohol abuse | African-American | T | 0.9 | | 0.0 | |
| | Caucasian | C | 0.6 | H | 1.0 | H |
| Alcoholic cirrhosis*⁴ | African-American | T | 1.0 | | 3.0 | H |
| Anxiety | African-American | T | 0.6 | | 0.0 | |
| | Caucasian | C | 0.5 | H | 1.0 | H |
| Asthma | African-American | C | 9.8 | H | 5.0 | H |
| | Caucasian | C | 0.5 | H | 1.0 | H |
| COPD | African-American | C | 3.0 | | 21.0 | |
| | Caucasian | T | 0.0 | | 0.3 | H |
| Cholecystectomy | African-American | T | 0.7 | | 0.0 | |
| | Caucasian | C | 0.5 | H | 1.0 | H |
| DJD | African-American | C | 1.6 | | 3.5 | |
| | Caucasian | C | 0.5 | H | 1.0 | H |
| ESRD and frequent | African-American | C | 1.8 | | 7.0 | |
| de-clots | Caucasian | C | 0.5 | H | 1.0 | H |
| ESRD due to FSGS | African-American | T | 0.4 | | 3.5 | |
| | Caucasian | C | 0.5 | H | 1.0 | H |
| ESRD due to IDDM | African-American | T | 0.4 | | 1.0 | |
| Seizure disorder | African-American | T | 1.1 | | 7.0 | |
| | Caucasian | C | 0.6 | H | 1.0 | H |

*Compared to HTN alone.
*¹Compared to MI with HTN.
*²Compared to NTDDM alone.
*³Compared to MI with NIDDM.
*⁴Compared to Alcohol abuse.

PCR and sequencing were conducted as described in Example 1. The primers used were those in Example 1. The control samples were in good agreement with Hardy-Weinberg equilibrium, as follows:

For the Group I diseases, a frequency of 0.84 for the C allele (“p”) and 0.16 for the T allele (“q”) among black male control individuals predicts genotype frequencies of 71% C/C, 26% C/T, and 3% T/T at Hardy-Weinberg equilibrium (p²+2pq+q²=1). The observed genotype frequencies were 79% C/C, 11% C/T, and 11% T/T, in fair agreement with those predicted for Hardy-Weinberg equilibrium.

A frequency of 0.92 for the C allele (“p”) and 0.08 for the T allele (“q”) among black female control individuals predicts genotype frequencies of 85% C/C, 14% C/T, and 1% T/T at Hardy-Weinberg equilibrium (p²+2pq+q²=1). The observed genotype frequencies were 84% C/C, 16% C/T, and 0% T/T, in very close agreement with those predicted for Hardy-Weinberg equilibrium.

A frequency of 1.0 for the C allele (“p”) and 0 for the T allele (“q”) among white male control individuals predicts genotype frequencies of 100% C/C, 0% C/T, and 0% T/T at Hardy-Weinberg equilibrium (p2+2pq+q²=1). The observed genotype frequencies were 100% C/C, 0% C/T, and 0% T/T, in perfect agreement with those predicted for Hardy-Weinberg equilibrium.

A frequency of 1.0 for the C allele (“p”) and 0 for the T allele (“q”) among white male control individuals predicts genotype frequencies of 100% C/C, 0% C/T, and 0% T/T at Hardy-Weinberg equilibrium (p²+2pq+q²=1). The observed genotype frequencies were 100% C/C, 0% C/T, and 0% T/T, in perfect agreement with those predicted for Hardy-Weinberg equilibrium.

For the Group II diseases, a frequency of 0.17 for the T allele (“p”) and 0.83 for the C allele (“q”) among African-American control individuals predicts genotype frequencies of 68.9% C/C, 28.2% C/T, and 2.9% T/T at Hardy-Weinberg equilibrium (p²+2pq+q²=1). The observed genotype frequencies were 82.0% C/C, 2.0% C/T, and 16.0% T/T, in distant agreement with those predicted for Hardy-Weinberg equilibrium.

A frequency of 0.0 for the T allele (“p”) and 1.0 for the C allele (“q”) among Caucasian control individuals predicts genotype frequencies of 100% C/C, 0% C/T, and 0% T/T at Hardy-Weinberg equilibrium (p²+2pq+q²=1). The observed genotype frequencies were 100% C/C, 0% C/T, and 0% T/T, in perfect agreement with those predicted for Hardy-Weinberg equilibrium.

Using an allele-specific odds ratio of 1.5 or greater as a practical level of significance (see Austin et al., discussed above), the following observations can be made.

For black men with hypertension, the odds ratio for the C allele as a risk factor for disease was 1.7 (95% CI, 0.3-9.2). The odds ratio for the homozygote (CC) was 2.7^{H} (95% CI, 0.3-25.4). The heterozygote (CT genotype) had a higher odds ratio of 5.0^{H} (95% C.I., 0.459.7). These data suggest that the C allele behaves as a co-dominant allele, since the heterozygote had almost a two-fold higher odds ratio than the homozygote.

For black women with hypertension, the odds ratio for the T allele as a risk factor for disease was 1.8 (95% CI, 0.3-10.0). The odds ratio for the homozygote (TT) was 1.9^{H} (95% CI, 0.1-33.0), while the heterozygote (TC genotype) had essentially the same odds ratio of 2.0 (95% CI, 0.3-12.2). These data suggest that the T allele behaves as a classical dominant allele.

For black men with ESRD due to HTN, the odds ratio for the C allele as a risk factor for disease was 2.1 (95% CI, 0.2-25.3) relative to black men with hypertension but no renal disease. The odds ratio for the homozygote (CC) was 3.1^{H} (95% CI, 0.3-28.3), while the heterozygote (CT genotype) had essentially the same odds ratio of 3.0^{H} (95% CI, 0.2-39.6). These data suggest that the C allele behaves as a classical dominant allele.

For black men with NIDDM, the odds ratio for the T allele as a risk factor for disease was 3.2 (95% CI, 0.6-17.1). The odds ratio for the homozygote (TT) was 2.1^{H} (95% CI, 0.2-24.0), whereas the heterozygote (TC genotype) had a much higher odds ratio of 22.5 (95% C.I., 1.5-335). These data suggest that the T allele behaves as a co-dominant allele.

For black women with NIDDM, the odds ratio for the T allele as a risk factor for disease was 1.9 (95% CI, 0.3-13.1). The odds ratio for the homozygote (TT) was 3.0^{H} (95% CI, 0.2-52.1), whereas the heterozygote (TC genotype) had a smaller odds ratio of 2.1 (95% CI, 0.3-16.6). These data suggest that the T allele behaves as a dominant allele, with an additive effect of allele dosage [3.0−2.1+2.1−1=3.2].

For white women with NIDDM, the odds ratio for the T allele as a risk factor for disease was 9.4H (95% Cl, 0.9-94.6). The odds ratio for the homozygote (TT) was 3.3^{H} (95% CI, 0.2-56.6), whereas the heterozygote (TC genotype) had a three-fold higher odds ratio of 9.9^{H} (95% C.I., 0.9-104). These data suggest that the T allele behaves as a co-dominant allele.

For black men with ESRD due to NIDDM, the odds ratio for the C allele as a risk factor for disease was 13.4^{H} (95% CI, 1.5-123) relative to black men with NIDDM but no renal disease. The odds ratio for the homozygote (CC) was 3.7^{H} (95% CI, 0.2-77.6), while the heterozygote (CT genotype) had an odds ratio of even less than 1^{H}. These data suggest that the C allele behaves in a recessive fashion.

For white women with ESRD due to NIDDM, the odds ratio for the C allele as a risk factor for disease was 2.3^{H} (95% CI, 0.2-24.1) relative to white women with NIDDM but no renal disease. The genotype-specific odds ratios are unfortunately uninformative, possibly due to distortion of the data by the Haldane's correction. Thus, no conclusion can be drawn regarding how the C allele contributes to diabetic nephropathy among white women.

For African-Americans with asthma the odds ratio for the C allele was 19.9^{H} (95% Cl, 1.2-340.6). The odds ratio for the homozygote (C/C) was 9.8 (95% CI, 0.6-167.1), while the odds ratio for the heterozygote (C/T) was 5.0 (95% CI, 0.1-366.3). These data suggest that the C allele acts in a dominant manner in this patient population with an approximately additive effect of allele dosage [9.8˜9.0=(5+5−1.0)]. (Goldstein et al., Monogr. Natl. Cancer Inst., 26:49-54, 1999). These data further suggest that the vHL gene is significantly associated with asthma in African-Americans, i.e. abnormal activity of the vHL gene predisposes African-Americans to asthma.

For African-Americans with cataracts due to HTN the odds ratio for the C allele was 2.7 (95% CI, 0.7-10). The odds ratio for the homozygote (C/C) was 7.8 (95% CI, 0.5-134), while the odds ratio for the heterozygote (C/T) was 35 (95% CI, 1.1-1094.8). These data suggest that the T allele acts in a co-dominant manner in this patient population. These data further suggest that the vHL gene is significantly associated with cataracts due to HTN in African-Americans, i.e. abnormal activity of the vHL gene predisposes African-Americans to cataracts due to HTN.

For Caucasians with cataracts due to HTN the odds ratio for the T allele was 6.4^{H} (95% CI, 0.3-160.4). The odds ratio for the homozygote (T/T) was 0.5 (95% CI, 0-7.9), while the odds ratio for the heterozygote (C/T) was 3.0 (95% CI, 0-473.1). These data suggest that the T allele acts in a co-dominant manner in this patient population. These data further suggest that the vHL gene is significantly associated with cataracts due to HTN in Caucasians, i.e. abnormal activity of the vHL gene predisposes Caucasians to cataracts due to HTN.

For Caucasians with COPD the odds ratio for the T allele was 100.9^{H} (95% CT, 0.5-232.8). Data were not sufficient to generate genotypic odds ratios of 1.5 or greater. These data further suggest that the vHL gene is significantly associated with COPD in Caucasians, i.e. abnormal activity of the vHL gene predisposes Caucasians to COPD.

For African-Americans with diabetic cardiomyopathy the odds ratio for the T allele was 2.7 (95% CI, 0.7-11.2), compared to African-Americans with MI due to NIDDM. The odds ratio for the homozygote (T/T) was 0.4 (95% CI, 0-4.9), while the odds ratio for the heterozygote (C/T) was 1.5 (95% CL 0.1-40.6). These data suggest that the T allele acts in a manner in this patient population. These data further suggest that the vHL gene is significantly associated with diabetic cardiomyopathy in African-Americans, i.e. abnormal activity of the vHL gene predisposes African-Americans to diabetic cardiomyopathy.

For African-Americans with ESRD due to IDDM the odds ratio for the T allele was 2.3 (95% CI, 1-5.2). Data were not sufficient to generate genotypic odds ratios of 1.5 or greater. These data further suggest that the vHL gene is significantly associated with ESRD due to IDDM in African-Americans, i.e. abnormal activity of the vHL gene predisposes African-Americans to ESRD due to IDDM.

For African-Americans with ESRD due to FSGS the odds ratio for the T allele was 2.6 (95% Cr, 1.1-6). The odds ratio for the homozygote (T/T) was 0.4 (95% CI, 0.11.4), while the odds ratio for the heterozygote (C/T) was 3.5 (95% CI, 0.3-43.2). These data suggest that the T allele acts in a co-dominant manner in this patient population. These data further suggest that the vHL gene is significantly associated with ESRD due to FSGS in African-Americans, i.e., abnormal activity of the vHL gene predisposes African-Americans to ESRD due to FSGS.

For African-Americans with hypertensive cardiomyopathy the odds ratio for the C allele was 3.8 (95% CI, 0.9-15.2), compared to African-Americans with MI due to HTN. The odds ratio for the homozygote (C/C) was 3.1 (95% CI, 0.3-38), while the odds ratio for the heterozygote (C/T) was 0.5 (95% CI, 0-12.9). These data suggest that the C allele acts in a recessive manner in this patient population. These data further suggest that the vHL gene is significantly associated with hypertensive cardiomyopathy in African-Americans, i.e. abnormal activity of the vHL gene predisposes African-Americans to hypertensive cardiomyopathy.

For African-Americans with CVA due to HTN the odds ratio for the T allele was 4.5 (95% CI, 1.4-15), compared to African-Americans with hypertension only. Data were not sufficient to generate genotypic odds ratios of 1.5 or greater. These data further suggest that the vHL gene is significantly associated with CVA due to HTN in African-Americans, i.e. abnormal activity of the vHL gene predisposes African-Americans to CVA due to HTN.

For African-Americans with MI due to NIDDM the odds ratio for the C allele was 3.7 (95% CI, 0.9-14.7), compared to African-Americans with NIDDM only. The odds ratio for the homozygote (C/C) was 3.9 (95% CT, 0.4-41.5), while the odds ratio for the heterozygote (C/T) was 1.0 (95% CI, 0-24.5). These data suggest that the C allele acts in a recessive manner in this patient population. These data further suggest that the vHL gene is significantly associated with MI due to NIDDM in African-Americans, i.e. abnormal activity of the vHL gene predisposes African-Americans to MI due to NIDDM.

For African-Americans with MI due to HTN the odds ratio for the T allele was 2.5 (95% CI, 0.7-9.1), compared to African-Americans with hypertension only. Data were not sufficient to generate genotypic odds ratios of 1.5 or greater. These data further suggest that the vHL gene is significantly associated with MI due to HTN in African-Americans, i.e. abnormal activity of the vHL gene predisposes African-Americans to MI due to HTN.

According to GENOMATIX MatInspector, the C638->T SNP potentially disrupts the binding site for the Wilm's Tumor transcription factor (WT1_B. as abbreviated by GENOMATIX; Nakagama et al., Mol. Cell Biol., 15:1489-1498, 1995). The consensus binding sequence for WT1_B consists of the 13 nucleotides 5′-GNGTGGGSGCGNS-3′ (SEQ ID NO: 7). This sequence occurs on the (−) strand of the vHL promoter. The C638->T SNP replaces the indicated G on the (−) strand with an A. This can be seen more easily as follows. The complement of this sequence, 5′-SNCGCSCCCACNC-3′ (SEQ ID NO: 8), occurs on the (+) strand (nucleotides 634-646, inclusive). The C638->T SNP replaces the indicated C with a T on the (+) strand. The complement of this T is an A on the (−) strand.

The WT1_B binding sequence matches nucleotides 634-646 on the (−) strand with a matrix score of 0.907 (where 1.000 represents a perfect match). WT1_B binding sites occur on average 0.97 times per 1000 base pairs of random vertebrate genomic DNA. The effect of the C638->T SNP is predicted to be weakening of the WT1_B binding site, although it is unknown whether WT1_B acts as a transcriptional activator or repressor of the vHL gene.

It is quite interesting that the C638->T SNP disturbs a binding site for the Wilm's Tumor gene product, which itself is a transcription factor and tumor suppressor specific for a kidney tumor (nephroblastoma). The vHL gene, of course, is also a tumor suppressor involved rather specifically in renal cell cancer. This is the only WT1_B binding site in the vHL promoter (nucleotides 1-643 of Accession Number AF010238).

A plausible arrangement, since the absence of vHL and WT1 activity both lead to unregulated growth, is that vHL and WT1 are involved coordinately in growth control of the kidney. The unique WT1_B site effected by the C638->T SNP suggests that WT1 acts as a transcriptional activator of the vHL gene. The effect of the T allele is therefore predicted to be a decrease in transcription of the vHL gene. The specificity of the interaction between WT1 and vHL suggests that this effect of the T allele may be a potent one.

TABLE 13

| | | | Reference | | |
|---|---|---|---|---|---|
| Gene | Region | Location | Type | Variant | SEQ ID |
| VHL | Promoter | 520 | A | G | 1 |
| | | 638 | C | T | 1 |

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for diagnosing a genetic susceptibility for a disease, condition, or disorder in a subject comprising:
obtaining a biological sample containing nucleic acid from said subject; and
analyzing said nucleic acid to detect the presence or absence of a single nucleotide polymorphism in the vHL gene, wherein said single nucleotide polymorphism is associated with a genetic predisposition for a disease, condition or disorder selected from the group consisting of colon cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, alcoholic cirrhosis, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to non-insulin dependent diabetes mellitus, end stage renal disease due to insulin dependent diabetes mellitus, and seizure disorder.

2. The method of claim 1, wherein the gene vHL comprises SEQ ID NO: 1.

3. The method of claim 1, wherein said nucleic acid is DNA, RNA, cDNA or mRNA.

4. The method of claim 2, wherein said single nucleotide polymorphism is located at position 520 or 638 of SEQ ID NO: 1.

5. The method of claim 4, wherein said single nucleotide polymorphism is selected from the group consisting of A520->G and C638->T and its complements namely T520->C and G638->A.

6. The method of claim 1, wherein said analysis is accomplished by sequencing, mini sequencing, hybridization, restriction fragment analysis, oligonucleotide ligation assay or allele specific PCR.

7. An isolated polynucleotide comprising at least 10 contiguous nucleotides of SEQ ID NO: 1, or the complement thereof, and containing at least one single nucleotide polymorphism at position 520 or 638 of SEQ ID NO: 1 wherein said at least one single nucleotide polymorphism is associated with a disease, condition or disorder selected from the group consisting of colon cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, alcoholic cirrhosis, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to non-insulin dependent diabetes mellitus, end stage renal disease due to insulin dependent diabetes mellitus, and seizure disorder.

8. The isolated polynucleotide of claim 7, wherein at least one single nucleotide polymorphism is selected from the group consisting of A520->G and C638->T and the complements thereof namely T520->C and G638->A.

9. The isolated polynucleotide of claim 7, wherein said at least one single nucleotide polymorphism is located at the 3′ end of said nucleic acid sequence.

10. The isolated polynucleotide of claim 7, further comprising a detectable label.

11. The isolated nucleic acid sequence of claim 10, wherein said detectable label is selected from the group consisting of radionuclides, fluorophores or fluorochromes, peptides, enzymes, antigens, antibodies, vitamins or steroids.

12. A kit comprising at least one isolated polynucleotide of at least 10 contiguous nucleotides of SEQ ID NO: 1 or the complement thereof, and containing at least one single nucleotide polymorphism associated with a disease, condition, or disorder selected from the group consisting of colon cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, alcoholic cirrhosis, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, end stage renal disease due to non-insulin dependent diabetes, and seizure disorder; and
instructions for using said polynucleotide for detecting the presence or absence of said at least one single nucleotide polymorphism in said nucleic acid.

13. The kit of claim 12 wherein said at least one single nucleotide polymorphism is located at position 520 or 638 of SEQ ID NO: 1.

14. The kit of claim 13 wherein said at least one single nucleotide polymorphism is selected from the group consisting of A520->G and C638->T and the complements thereof, namely T520->C and G638->A.

15. The kit of claim 12, wherein said single nucleotide polymorphism is located at the 3′ end of said polynucleotide.

16. The kit of claim 12, wherein said polynucleotide further comprises at least one detectable label.

17. The kit of claim 16, wherein said label is chosen from the group consisting of radionuclides, fluorophores or fluorochromes, peptides enzymes, antigens, antibodies, vitamins or steroids.

18. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease, condition, or disorder selected from the group consisting of colon cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, alcoholic cirrhosis, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, end stage renal disease due to non-insulin dependent diabetes mellitus, and seizure disorder; and instructions for using said polynucleotide for detecting the presence or absence of said single nucleotide polymorphism in a biological sample containing nucleic acid.

19. The kit of claim 18, wherein said single nucleotide polymorphism site is located at position 520 or 638 of SEQ ID NO: 1.

20. The kit of claim 19, wherein said at least one polynucleotide further comprises a detectable label.

21. The kit of claim 20, wherein said detectable label is chosen from the group consisting of radionuclides, fluorophores or fluorochromes, peptides, enzymes, antigens, antibodies, vitamins or steroids.

22. A method for treatment or prophylaxis in a subject comprising:
obtaining a sample of biological material containing nucleic acid from a subject;
analyzing said nucleic acid to detect the presence or absence of at least one single nucleotide polymorphism in SEQ ID NO: 1 or the complement thereof associated with a disease, condition, or disorder selected from the group consisting of colon cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, alcoholic cirrhosis, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, end stage renal disease due to non-insulin dependent diabetes, and seizure disorder; and
treating said subject for said disease, condition or disorder.

23. The method of claim 22 wherein said nucleic acid is selected from the group consisting of DNA, cDNA, RNA and mRNA.

24. The method of claim 22, wherein said at least one single nucleotide polymorphism is located at position 520 and 638 of SEQ ID NO: 1.

25. The method of claim 22 wherein said at least one single nucleotide polymorphism is selected from the group consisting of A520->G and C638->T and the complements thereof, namely T520->C and G638->A

26. The method of claim 22 wherein said treatment counteracts the effect of said at least one single nucleotide polymorphism detected.
